SEQUENCE LISTING (4 sequences)

SEQ ID NO: 1 - protein, 347 aa, Artificial Sequence
  Human Leucine-rich alpha-2-glycoprotein
MSSWSRQRPKSPGGIQPHVSRTLFLLLLLAASAWGVTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 2 - protein, 312 aa, Artificial Sequence
  Human Leucine-rich alpha-2-glycoprotein_active sequence
VTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 3 - RNA, 19 nt, Artificial Sequence
  shRNA_human LPHN2
acggcttatgggtcattta

SEQ ID NO: 4 - RNA, 19 nt, Artificial Sequence
  shRNA_mouse LPHN2
tcaactgctaggtcgatat

(19) United States
(12) Patent Application Publication
(10) Pub. No.: US 2024/0083959 A1
(43) Pub. Date: Mar. 14, 2024

(54) DEGLYCOSYLATED LRG1 GLYCOPROTEIN AND LRG1 GLYCOPROTEIN VARIANT, AND USE THEREOF

(71) Applicants: INSTITUTE FOR BASIC SCIENCE, Daejeon (KR); INHA UNIVERSITY RESEARCH AND BUSINESS FOUNDATION, Incheon (KR); KOREA ADVANCED INSTITUTE OF SCIENCE AND TECHNOLOGY, Daejeon (KR)

(72) Inventors: Ho Min KIM, Daejeon (KR); Do-Kyun KIM, Daejeon (KR); Ji-Kan RYU, Incheon (KR)

(21) Appl. No.: 18/273,080

(22) PCT Filed: Jan. 24, 2022
(86) PCT No.: PCT/KR2022/001179

§ 371 Date: Jul. 19, 2023

(30) Foreign Application Priority Data
Jan. 22, 2021 (KR) 10-2021-0009214

(51) Int. Cl. C07K 14/47

(52) U.S. Cl. CPC C07K 14/473; A61K 38/00

(57) ABSTRACT

The present invention relates to a deglycosylated LRG1 glycoprotein and a LRG1 glycoprotein variant, and a use thereof. The deglycosylated LRG1 glycoprotein and LRG1 glycoprotein variant of the present invention exhibit effects of angiogenesis, nerve growth and nerve regeneration that are superior to those of conventional LRG1 glycoproteins, and thus a composition containing thereof is effective in preventing and treating vascular erectile dysfunction, ischemic heart/brain/peripheral vascular diseases, diabetic vascular complications, neurogenic erectile dysfunction, diabetic neurologic complications, post-operative/post-traumatic peripheral nerve damage, ischemic diseases including neurodegenerative diseases, peripheral nerve diseases, erectile dysfunction and/or neurodegenerative diseases.

### TECHNICAL FIELD

The present invention relates to a deglycosylated LRG1 glycoprotein, a LRG1 glycoprotein variant, and the use thereof, wherein the deglycosylated LRG1 glycoprotein or LRG1 glycoprotein variant binds to and interacts with LPHN2, induces phosphorylation of Akt and NF-kB p65, and thus is effective in inducing high angiogenesis, neurotization and neurogenesis.

### BACKGROUND ART

Leucine-rich α-2 glycoprotein (LRG1), which was first isolated from the serum of a healthy person, was reported to be abundant at a level of 10 to 50 μg/ml in human serum (Haupt and Baudner, 1977; Takahashi et al., 1985). LRG1 is a member of highly conserved member of leucine-rich repeat (LRR) family of proteins (Bella et al., 2008; Ng et al., 2011).

Since LRG1 was first discovered in serum, it has been identified to be differentially expressed in inflammatory diseases (Serada et al., 2012), hydrocephalus (Nakajima et al., 2011), heart failure (Watson et al., 2011), autoimmune diseases (Serada et al., 2010), neurodegenerative diseases (Miyajima et al., 2013), and various types of cancer such as ovarian cancer, lung cancer, cholangiocarcinoma, and hepatocellular carcinoma (Andersen et al., 2010; Ladd et al., 2012; Okano et al., 2006; Sandanayake et al., 2011) and thus has received attention as a prognostic/diagnostic biomarker for these diseases. However, little has been reported about the pathological mechanisms and physiological roles of LRG1 in various diseases in which LRG1 is differentially expressed.

Recently, LRG1 exhibits highly upregulated expression in mouse retina in addition to choroidal and retinal angiogenesis, and upregulated LRG1 binds to endoglin (ENG), which is a TGF-β co-receptor, and activates the Smad1/5/8 pathway to promote pathological angiogenesis in the presence of TGF-β that plays an important role in the formation and functions of endothelial cells (X. Wang et al. Nature 499, 306-311 (2013)). However, LRG1 and ENG have relatively weak binding affinity (˜2.9 μM), compared to typical receptor-ligand interactions. Therefore, there is a limitation in explaining the physiology of LRG1 only with the mechanism of activating the TGF-β receptor through the interaction of LRG1 with TGF-β and ENG.

Meanwhile, diabetes mellitus (DM) is a chronic metabolic disorder and incidence rates thereof are increasing rapidly worldwide. Diabetes is characterized by hyperglycemia due to defects in insulin secretion and/or insulin resistance. Diabetes patents have symptoms such as endothelial dysfunction and neuropathy, which lead to multiple complications, including erectile dysfunction, cardiovascular diseases, stroke, chronic kidney diseases, foot ulcers, and retinopathy (Int J Vasc Med 2012, 918267 (2012)). Diabetic patients also have high LRG1 plasma levels, which is associated with complications such as vascular endothelial dysfunction, atherosclerosis and peripheral arterial disease, and poor prognosis for diabetic kidney diseases (J Am Soc Nephrol 30, 546-562 (2019)), and corresponds to the phenotype of a mouse model of pathogenic TGF-β/ALK1-mediated angiogenesis (Journal of Clinical Endocrinology and 40 Metabolism 100, 1586-1593 (2015)). In contrast, exogenous corneal treatment of streptozotocin (STZ)-induced diabetic mouse models with LRG1 accelerates epithelial wound healing and nerve regeneration through TGF-β-dependent signaling (Li et al., 2020).

Erectile dysfunction (ED) is often found in diabetes mellitus and the pathophysiology of diabetic erectile dysfunction leads to microangiopathy such as ischemia and neuropathy such as impaired axon regeneration in peripheral nerves (Kolluru et al., 2012). The association of LRG1 with erectile dysfunction, angiopathy, and neuropathy in diabetic patients has not been identified at all.

Recent research on erectile dysfunction focuses on organic causes, and oral PDE-5 (phosphodiesterase-5) inhibitors, including Viagra®, are used all over the world, but are ineffective in about 30% of patients and ultimately fail to treat angiopathy and neuropathy in patients with diabetic erectile dysfunction (Kim et al., 2020).

Considering the fact that blood vessels and nerves have similar alignment patterns and are aligned in parallel only in some sites (Nature 436, 193-200 (2005)), targeting a common mechanism in the formation of nerve and vascular networks is recognized as a highly effective strategy for simultaneous treatment of vasculopathy and neuropathy. Factors such as VEGF, angiopoietin, NGF, BDNF, FGF2, and HGF, which are called “angioneurins”, are involved in the formation and development of blood vessels and nerves, and damage of the pathways of these factors may cause both neurological and vascular disorders. Various angiogenic or neurotrophic factors such as COMP-Ang1, vascular endothelial growth factors (VEGFs), dickkorf2, neurotrophin-3 (NT3) and brain-derived neurotrophic factors (BDNFs) have been tested for the treatment of angiopathy and neuropathy (Bennett et al., 2005; Burchardt et al., 2005; Ghatak et al., 2017; Hu et al., 2018; Jin et al., 2011), but substances that had clinical success are very limited.

Under this technical background, as a result of extended efforts to develop a novel protein drug for preventing or treating diabetic erectile dysfunction, ischemic diseases, and neurological diseases, the present inventors found that the fusion protein of LRG1 and Fc domain induces regeneration of penile corpus cavernosum endothelial cells and nerve cells, increases penile erection, and exhibits excellent blood vessel and nerve regeneration effects, based on the fact that blood vessels and nerves can be regenerated through the interaction of LRG1 glycoprotein and TGF-β/ENG, and registered a patent relating to the use of fusion proteins for erectile dysfunction, ischemic diseases and neurological diseases based thereon (Korean Patent Nos. 2162934 and 2002866). However, due to the weak binding affinity between LRG1 and ENG (˜2.9 μM), it is difficult to fully explain the vascular and nerve regenerative effects of LRG1 only with TGF-β-based mechanisms.

Under this background art, the present inventors predicted that there would be another vascular and neurogenic pathway of LRG1, in addition to the TGF-β/ENG-based pathway, and made diligent efforts to verify this prediction. As a result, the present inventors found that LRG1 exhibits TGF-β-independent angiogenic and neurotrophic effects under hyperglycemic conditions such as diabetic patients and identified TGF-β-independent vascular and nerve regeneration mechanisms based on LPHN2 (latrophilin-2) interactions and downstream signaling. Furthermore, the present inventors found that deglycosylation of LRG1 under hyperglycemic conditions plays a critical role in binding to LPHN2, and administration of artificially deglycosylated LRG1 directly (through LPHN2 pathway) and indirectly (by enhancement of neurotrophic factor expression in nerve cells). The present invention was completed based thereon.

The information disclosed in this Background section is provided only for better understanding of the background of the present invention, and therefore it may not include information that forms the prior art that is already obvious to those skilled in the art.

### DISCLOSURE

### Technical Problem

Therefore, the present invention has been made in view of the above problems, and it is one object of the present invention to provide a deglycosylated LRG1 glycoprotein that is highly effective in inducing angiogenesis, neurotization and neurogenesis.

It is another object of the present invention to provide a novel LRG1 receptor and a TGF-β-independent angiogenesis and blood vessel growth mechanism based on the LRG1 receptor.

It is another object of the present invention to provide an LRG1 glycoprotein containing a variant at a glycosylation site.

It is another object of the present invention to provide a fusion protein in which the deglycosylated LRG1 glycoprotein or LRG1 glycoprotein variant and a Fc domain are fused with each other.

It is another object of the present invention to provide the use of the deglycosylated LRG1 glycoprotein, the LRG1 glycoprotein variant, and the fusion protein.

### Technical Solution

In accordance with one aspect of the present invention, the above and other objects can be accomplished by the provision of a LRG1 glycoprotein (leucine rich α-2 glycoprotein) in which at least one glycosyl group is deglycosylated.

In accordance with another aspect of the present invention, provided is an LRG1 glycoprotein (leucine rich α-2 glycoprotein) variant including at least one variation in at least one glycosylation site.

In accordance with another aspect of the present invention, provided is a nucleic acid encoding the LRG1 glycoprotein variant.

In accordance with another aspect of the present invention, provided is a recombinant vector including the nucleic acid.

In accordance with another aspect of the present invention, provided is a host cell into which the nucleic acid or the recombinant vector is introduced.

In accordance with another aspect of the present invention, provided is a method of producing an LRG1 glycoprotein variant including culturing the host cell to produce an LRG1 glycoprotein variant and obtaining the produced LRG1 glycoprotein variant.

In accordance with another aspect of the present invention, provided is a fusion protein in which an Fc domain is fused to the LRG1 glycoprotein or the LRG1 glycoprotein variant.

In accordance with another aspect of the present invention, provided is a composition for inducing angiogenesis, neurogenesis and neurotization containing the LRG1 glycoprotein, the LRG1 glycoprotein variant, or the fusion protein.

In accordance with another aspect of the present invention, provided is a composition for preventing or treating ischemic diseases, peripheral nerve diseases, erectile dysfunction and/or neurodegenerative diseases containing the LRG1 glycoprotein, the LRG1 glycoprotein variant, or the fusion protein.

In accordance with another aspect of the present invention, provided is a method for inducing angiogenesis, neurogenesis and/or neurotization including administering the LRG1 glycoprotein, the LRG1 glycoprotein variant, or the fusion protein to a subject.

In accordance with another aspect of the present invention, provided is a method for treating ischemic diseases, peripheral nerve diseases, erectile dysfunction and/or neurodegenerative diseases including administering the LRG1 glycoprotein, LRG1 glycoprotein variant, or fusion protein to a subject.

In accordance with another aspect of the present invention, provided is the use of the LRG1 glycoprotein, the LRG1 glycoprotein variant, or the fusion protein for inducing angiogenesis, neurogenesis and/or neurotization.

In accordance with another aspect of the present invention, provided is the use of the LRG1 glycoprotein, the LRG1 glycoprotein variant, or the fusion protein for treating ischemic diseases, peripheral nerve diseases, erectile dysfunction and/or neurodegenerative diseases.

In accordance with another aspect of the present invention, provided is the use of the LRG1 glycoprotein, the LRG1 glycoprotein variant, or the fusion protein for the preparation of a composition for inducing angiogenesis, neurogenesis and/or neurotization.

In accordance with another aspect of the present invention, provided is the use of the LRG1 glycoprotein, the LRG1 glycoprotein variant, or the fusion protein for the preparation of a composition for treating ischemic diseases, peripheral nerve diseases, erectile dysfunction and/or neurodegenerative diseases.

### DESCRIPTION OF DRAWINGS

FIG. 1 illustrates that LPHN2, an adherent GPCR, is a TGF-β-independent receptor for LRG1.

FIG. 1A: a schematic diagram illustrating a LRC-TriCEPS for identifying the TGF-β-independent receptor of LRG1.

FIG. 1B: a volcano plot illustrating FDR-adjusted P-values plotted for fold change (Fold, Change; FC) between samples by comparing TriCEPS-bound LRG1 or transferrin with a glycine quenched TriCEPS reagent control sample, wherein three proteins, namely, LPHN2, LEG3, and NDUA5, are receptor candidates defined by an enrichment factor greater than 4 and an FDR-adjusted P-value less than 0.05.

FIG. 1C: cell surface binding of LRG1-YFP or YFP to parental (shCon-expressing) or LPHN2-knockdown HEK293T cells (top) and HUVECs (bottom). Scale bar, 100 μm.

FIG. 1D: Immunoprecipitation (IP) of LPHN2 from whole HUVEC lysates. LRG1 and LPHN2 were detected through immunoblot analysis after treatment for 10 minutes, regardless of the presence of LRG1 (1 μg/ml). Data are expressed as mean±SEM (n=3).

FIGS. 1E and 1F: Ex vivo mouse aortic ring assay (E) and mouse cavernous endothelial cell (MCEC) sprouting assay (F) in mice treated with the indicated proteins and lentivirus (shCon, shLPHN2) or anti-TGF-β1 antibody under normal glucose (NG) or high-glucose (HG) conditions. Sprouted microvessels were immunostained with PECAM-1 (red, E), an endothelial cell marker. The dotted line represents the range of sprouted endothelial cells (F). Scale bar, 100 μm. The intensity of microvessel areas sprouting in aortic rings (mean±SEM (n=4), E) and endothelial cells sprouting in spongy tissues (mean±SEM (n=6), F) were quantified (bottom). The relative ratio of the NG group was defined as 1.

FIG. 2 shows the result of identification of a novel receptor to which LRG1 binds in the absence of TGF-β1.

FIGS. 2A and 2B: Tube formation assay (A) and transwell cell migration assay (B) using HUVECs performed after treatment with indicated concentrations of the following proteins: Fc (1 μg/ml; negative control), LRG1-Fc (1 μg/ml), TGF-β1 (5 ng/ml), LRG1-Fc (1 μg/ml)+TGF-β1 (5 ng/ml), LRG1-Fc (1 μg/ml)+anti-TGF-β1 Ab (10 μg/ml) and LRG1-Fc (1 μg/ml)+TGF-β1 (5 ng/ml)+anti-TGF-β1 Ab (10 μg/ml). Left: representative image, scale bar, 200 μm. Right: Master junctions and migrated cells were quantified using Image J and the results are expressed as mean±SEM (n=4). The relative ratio of the Fc group was defined as 1 (B).

FIG. 2C: Cell surface binding of LRG1-YFP or YFP to HEK293T cells (left) and HUVECs (right). Evaluation was performed after LRG1-YFP (10 μg/ml) or YFP (10 μg/ml) treatment, and nuclei were labeled with 4,6-diamidino-2-phenylindole (DAPI; blue). Scale bar, 100 μm.

FIG. 2D: conjugation of ligands, including the negative control glycine, positive control transferrin and LRG1, with the biotin-containing TriCEPS reagent using NHS ester functionality thereof. Binding of TriCEPS-conjugated ligands to cell surface receptors was detected by FITC-streptavidin and analyzed by FACS. The result of analysis showed that transferrin-TriCEPS and LRG1-TriCEPS bound to HEK293T cells, without interference from the TriCEPS moiety.

FIG. 2E: results of Western blots to detect LPHN2 expression in HEK293T cells and HUVECs treated with shCon or shLPHN2 lentivirus.

FIG. 2F: results obtained by treating HUVECs with a LRG1 protein (1 μg/ml) at indicated time intervals, immunoprecipitation (IP) of LPHN2 from whole-cell lysates, resolution on SDS-PAGE gels, and staining with Coomassie Blue solution.

FIG. 2G: Identification of LPHN2-interacting proteins by LC-MS/MS analysis, wherein gel bands for LC-MS/MS analysis (˜70 kDa) are shown in FIG. 2F and proteins are listed in accordance with the top matching peptides.

FIG. 2H: Tube-formation assay. MCECs under high-glucose (HG) conditions were incubated with PBS (negative control) or LRG1 (1 μg/ml) for 72 hours, and then tube formation was assayed. Control (shCon) and LPHN2-knockdown (shLPHN2) lentiviruses were added to culture medium at 5×10⁴ transduction units (TU)/ml. Left: representative images of tube formation. Scale bars, 100 μm. Right: master junctions were quantified using Image J and the results are expressed as means±SEM (n=4).

FIG. 2I: MCECs were infected with lentivirus containing control shRNA (shCon) or shRNA targeting LPHN2 (shLPHN2) at two different doses (5×10³ TU and 5×10⁴ TU/ml culture medium) for at least 72 hours. Top: Representative Western blots for LPHN2 from MCECs infected with shCon or shLPHN2 lentivirus. Bottom: Normalized band intensity values were quantified using Image J and the results are expressed as means±SEM (n=4). The relative ratio of the shCon group was defined as 1.

FIG. 2J: Representative Western blots for TGFβ1 in conditioned medium from MCECs (left), aorta ring tissue (middle), and cavernosum tissue (right) under NG and HG conditions with increased TGFβ1 levels in culture medium under HG conditions. Normalized band intensity values were quantified using Image J and the results are expressed as means±SEM (n=4, bottom). The relative ratio of the NG group was defined as 1.

FIG. 3 illustrates that LRG1/LPHN2-mediated angiogenesis ameliorates erectile dysfunction in STZ-induced diabetic mice.

FIGS. 3A and 3B: Representative intracavernous pressure (ICP) responses (A) and immunostaining (B, platelet/endothelial cell adhesion molecule-1 (PECAM-1, green) and neuro-glial antigen 2 (NG2, red))) in cavernous tissue in the control and STZ-induced diabetic mice at 2 weeks after repeated intracavernous injections of Fc (negative control, 5 μg/20 μl), LRG1-Fc (5 μg/20 μl), or LRG1-Fc (5 μg/20 μl)+Anti-TGF-β1 antibody (10 μg/ml) at day 0 and 3 under scramble shRNA control (shCon) and LPHN2 knockdown shRNA (shLPHN2) (1×10⁴ TU/mouse) conditions. (A) the stimulus interval is represented by a solid bar. The ratios of mean maximal ICP to mean systolic blood pressure (MSBP) and total ICP (area under the curve) to mean systolic blood pressure (MSBP) were calculated for each group. (B) the relative ratio of the shCon+Fc group was defined as 1 (n=5, right).

FIGS. 3C to 3H: Immunostaining of phosphorylated eNOS (p-eNOS) (C), BrdU (E), Claudin-5 (F), oxidized-low-density lipoprotein (LDL, G), and TUNEL (H) in cavernous tissue from STZ-induced diabetic mice 2 weeks after repeated intracavernous injections of Fc or LRG1-Fc. Scale bars, 100 μm

FIG. 3D: Representative Western blots showing p-eNOS and eNOS in whole penile tissue under the conditions described above.

FIG. 3I: Quantification of p-eNOS expression by immunostaining and Western blotting (n=4), the number of BrdU immunopositive (+) endothelial cells per high-power field (n=4), claudin-5 (n=4) and oxidized-LDL (n=4), and the number of TUNEL (+) apoptotic endothelial cells per high-power field (n=4).

*P<0.05; **P<0.01; ***P<0.001 (student's t test). N.S.: Not significant. [65]

FIG. 4 illustrates the angiogenic effect of LRG1 in STZ-induced LRG1-Tg mice.

FIG. 4A: Representative intracavernous pressure (ICP) responses at 8 weeks in LRG1-Tg mice, and LRG1-Tg mice receiving STZ injection under scramble shRNA control (shCon) or LPHN2 knockdown shRNA (shLPHN2) condition (1×10⁵ TU/mouse) (top). The cavernous nerve was stimulated at 5 V. The stimulus interval is indicated by a solid bar. Ratios of mean maximal ICP to mean systolic blood pressure (MSBP) and total ICP (area under the curve) to mean systolic blood pressure (MSBP) were calculated for each group (n=5).

FIG. 4B: results of double-immunostaining of cavernous tissues of STZ-induced LRG1-Tg mice and LRG1-Tg mice injected with shCon or shLPHN2 lentivirus (1×10⁵ TU/mouse) with PECAM-1 (green) and claudin-5 or oxidized low-density lipoprotein (LDL). Claudin-5 and oxidized LDL expression were quantified using Image J (n=4), and the relative ratio of the LRG1-Tg group was defined as 1. Scale bar, 100 μm.

*P<0.05; **P<0.01; ***P<0.001 (student's t test). N.S.: Not significant.

FIG. 5 illustrates a LPHN2-dependent neurotrophic effect of LRG1.

FIG. 5A: results of staining of LPHN2 (red) and nerve fibers (NF; green) in mouse corpus cavernosum tissue (top) and dorsal nerve bundle (DNB; high magnification, bottom). Nuclei were stained with 4,6-diamidino-2-phenylindole (DAPI; blue). Scale bars, 200 μm (top) and 25 μm (bottom).

FIG. 5B shows expression of LPHN2 (mean±SEM (n=6)) in dorsal nerve bundles of wild-type (WT) and STZ-induced diabetic mice.

FIG. 5C: results of immunostaining of BIII-tubulin (green) in dorsal nerve bundle (DNB) tissue in the control and STZ-induced diabetic mice at 2 weeks after repeated intracavernous injections of Fc (negative control, 5 μg/20 μl), or LRG1-Fc (5 μg/20 μl) at day 0 and 3 under scramble shRNA control (shCon) and LPHN2 knockdown shRNA (shLPHN2) (1×10⁵ TU/mouse) conditions (mean±SEM (n=6)). In FIGS. 5B and 5C, nuclei were stained with DAPI (blue).

FIG. 5D: results of immunostaining of βIII-tubulin (red) and nNOS (green) in dorsal nerve bundle (DNB) tissue in the control and STZ-induced diabetic mice at 2 weeks after repeated intracavernous injections of Fc (negative control, 5 μg/20 μl), or LRG1-Fc (5 μg/20 μl) at day 0 and 3 (mean (n=4)). In FIGS. 5B, 5C and 5D, the relative ratio of the control group was defined as 1. Scale bar, 25 μm.

FIG. 5E: LPHN2 expression in mouse DRG tissues in normal glucose (NG) and high glucose (HG) conditions (mean±SEM (n=4, bottom)).

FIG. 5F: results of immunostaining of βIII-tubulin (top) in mouse DRG tissue treated with Fc (negative control, 1 μg/ml) or LRG1-Fc (1 μg/ml) for 1 week under normal-glucose (NG) or high-glucose (HG) conditions under the presence of scramble shRNA control (shCon) and LPHN2 knockdown shRNA (shLPHN2) (1×10⁵ TU/mouse) conditions. Scale bar, 100 μm. Quantification of BIII-tubulin-immunopositive neurite length in DRG tissue (bottom, mean±SEM (n=4)). The relative ratio of the NG group was defined as 1.

**P<0.01; ***P<0.001 (Student's t test). N.S.: Not significant.

FIG. 6 illustrates LPHN2 expression in STZ-induced diabetic mouse model and the neurotrophic effect of LRG1 in hyperglycemia.

FIG. 6A: expression of LPHN2 in mouse corpus cavernosum (CC) tissue. Top left: Mouse penile tissue was sectioned for immunofluorescence staining; Only CC tissue was used for the ex vivo MCEC sprouting assay. Left: representative LPHN2 (red) staining in spongy tissue from a normal mouse. Scale bar, 200 μm. Right: High magnification images of LPHN2 staining in corpus cavernosum (CC; scale bar, 100 μm), dorsal vein (DV; scale bar, 50 μm), cavernous artery (CA; scale bar, 25 μm) and dorsal artery (DA; scale bar, 25 μm). Nuclei were stained with DAPI (blue).

FIG. 6B: Increased LPHN2 expression in mouse penis, MCEC and MPG under STZ-induced diabetes and HG conditions. Top: Representative western blot for LPHN2. Bottom: normalized band intensity values were quantified using Image J and the results are expressed as means±SEM (n=4). The relative ratio of the NG control was defined as 1.

FIG. 6C: Immunostaining of cancellous tissues of control and STZ-induced diabetic mice with LPHN2 (red) and PECAM-1 (green). LPHN2 and PECAM-1 immunopositive areas in cancellous tissues were quantified using Image J and results are expressed as mean±SEM (n=6). Scale bar, 100 μm. The relative ratio of the control was defined as 1.

FIG. 6D: results of immunostaining of βIII-tubulin (green) in dorsal nerve bundle (DNB) tissue of wild-type (WT) control and LRG1-Tg mice after STZ injections for 8 weeks. BIII-tubulin immunopositive areas in the dorsal nerve bundle were quantified using Image J and results are expressed as mean±SEM (n=4). Scale bar, 25 μm. The relative ratio of the WT was defined as 1.

FIG. 6E: results of immunoostaining of βIII-tubulin for 1 week in mouse MPG tissue treated with Fc (negative control, 1 μg/ml) or LRG1-Fc (1 μg/ml) in the presence of scramble shRNA control (shCon), LPHN2 knockdown shRNA (shLPHN2) (5×10⁴ TU/ml culture medium) or anti-TGF-β1 antibody (10 μg/ml) under normal glucose(NG) or high glucose (HG). Scale bar, 100 μm. Bottom: quantification of βIII-tubulin-immunopositive neurite length in DRG tissue (mean±SEM (n=4)). The relative ratio of the NG group was defined as 1.

FIG. 6F: Increased TGFβ1 expression in culture medium under HG conditions. Representative Western blots for TGFβ1 in MPG tissue (left) and DRG tissue (right) in conditioned medium under RG and HG conditions. Normalized band intensity values were quantified using Image J and the results are expressed as means±SEM (n=4). The relative ratio of the NG group was defined as 1.

*P<0.05; **P<0.01; ***P<0.001 (student's t test). N.S.: Not significant. [88]

FIG. 7 illustrates the effect of increased binding of deglycosylated LRG1 to LPHN2 on improvement of angiogenesis and neurite outgrowth.

FIG. 7A: results of SDS-PAGE and Coomassie blue staining of samples of LRG1 cultured in HUVEC culture medium containing glucose-free, normal glucose or high glucose (G/F, NG, HG).

FIG. 7B: binding affinity of LRG1 (wild-type and deglycosylated forms) to LPHN2 ectodomains (Lec, Olf, Lec+Olf or Ecto-full domain; Lec, lectin; Olf, olfactomedin-like; HomoR, hormone receptor motif; GAIN/GPS, GPCR autoproteolysis-induced/GPCR proteolysis sites)

FIG. 7C: Results of FACS analysis of the binding of LRG1 to parental and LPHN2-knockdown HUVECs.

FIG. 7D: Tube formation assay (top) and transwell cell migration assay (bottom) using HUVECs treated with LRG1 (1 μg/ml) or DG-LRG1 (1 μg/ml). Scale bar, 200 μm.

FIG. 7E: Results of βIII-tubulin immunostaining in DRG tissues and cortical neurons (bottom) of mice treated with LRG1 (1 μg/ml) or DG-LRG1 (1 μg/ml). Scale bars, 200 μm for DRG and 100 μm for cortical neurons.

FIG. 7F: Results of quantification of the length of βIII-tubulin-immunopositive neurites in master junctions of HUVEC tube formation, migrated HUVECs, DRG explants and cortical neurons (200 cells/field) (mean±SEM (n=4), right). The relative ratio of the untreated group was defined as 1.

*P<0.05; **P<0.01; ***P<0.001 (student's t test). N.S.: Not significant. [97]

FIG. 8 illustrates the results of biochemical analysis of LRG1 and LPHN and sequence alignment of LPHN.

FIG. 8A: results of SDS-PAGE and Coomassie blue staining of glucose-free HUVEC-conditioned medium (G/F CM), normal glucose HUVEC-conditioned medium (NG CM) and high glucose HUVEC-conditioned medium (HG CM)

FIG. 8B: Schematic diagram of the domain structure of LPHN2 (left, abbreviations: Lec, lectin; Olf, olfactomedin-like; HomoR, hormone receptor motif; GAIN/GPS, GPCR self-proteolysis induction/GPCR proteolysis site). Purified recombinant human LPHN2 ectodomain variant (lectin domain, residues F26-Q95; olfactomedin-like domain, residues V135-P394; lectin-olfactomedin-like domain, residues F26-P394; and lectin-olfactomedin-like-GAIN/GPS domain, residues F26-R796) were analyzed by SDS-PAGE and Coomassie blue staining (right).

FIG. 8C: Binding affinity of LRG1 to HEK293 cells. Parental and LPHN2-knockdown HEK293T cells were treated with Alexa 647-conjugated LRG1 (1 μM) or DG-LRG1 (1 μM). After washing with PBS, fluorescence signals were measured by FACS to determine LRG1 and DG-LRG1 binding to cells.

FIG. 8D: Results of analysis of SDS-PAGE and Coomassie Blue staining of purified recombinant Lectin-Olfactomedin-like domains of human LPHN1 and LPHN3.

FIG. 8E: results of solid phase binding assay to evaluate binding of wild-type LRG1 and DG-LRG1 to the Lec-Olf domains of LPHN1 and LPHN3. The plates were coated with Lec-Olf domains (100 nM) of LPHN1 and LPHN3. After washing and blocking, different amounts (0.001, 0.005, 0.01, 0.05, 0.1, 0.5, 1, 5 and 10 μM) of wild-type LRG1 or DG-LRG1 were added and LRG1 or DG-LRG1 bound to coated proteins were detected by ELISA using anti-LRG1 antibody and anti-mouse secondary antibody.

FIG. 8F: results of SDS-PAGE and Coomassie blue staining of purified LRG1 variants (N269D, N325D, N269D/N325D) and control LRG1 (wild-type and deglycosylated forms).

FIGS. 8G and 8H: Tube formation assay using HUVECs (G) and transwell cell migration assay (H) after treatment with LRG1 (1 μg/ml) or DG-LRG1 (1 μg/ml) in the presence of shLPHN2 lentivirus (5×10⁴ TU/ml culture medium) or anti-TGF-β1 antibody (10 μg/ml). HUVECs were cultured in normal glucose medium. Scale bar, 200 μm.

FIGS. 81 and 8J: Results of βIII-tubulin immunostaining in mouse DRG tissue (I) and mouse primary cortical neurons (J) after treatment with LRG1 (1 μg/ml) or DG-LRG1 (1 μg/ml) in the presence of shLPHN2 lentivirus (5×10⁴ TU/ml culture medium) or anti-TGF-β1 antibody (10 μg/ml). Mouse DRG tissue and cortical neurons were cultured in normal glucose medium. Scale bars: 200 μm for (I) and 100 μm for (J).

FIG. 9 illustrates identification of the crystal structure of LRG1 and major glycosylation sites for the functions of LRG1.

FIG. 9A: Overall structure of human LRG1 (top view and side view). LRRNTs (blue); 8 LRR modules (green); LRRCT (orange); disulfide bridges (grey); phenylalanine spine and asparagine ladder (purple, magenta).

FIG. 9B: Glycans (yellow) attached to human LRG1 (turquoise surface). Bottom panel: Electron density map countered at 26 for N-acetylglucosamine attached to asparagine residues (N79, N186, N269 and N325).

FIG. 9C: Sequence alignment of glycosylation sites in LRG1. Strict equality (red box with white text); similarity in groups (red letters); similarity between groups (black letters). Glycosylated asparagine residues are represented by yellow hexagons.

FIG. 9D: Results of tube formation assay using HUVECs after treatment with 1 μg/ml of LRG1, DG-LRG1 or LRG1 variants (N79D, N186D, N269D, N325D). Scale bar, 200 μm. Right: quantification of master junctions (mean±SEM (n=4)).

FIGS. 9E and 9F: Results of βIII-tubulin immunostaining in mouse DRG tissue (E) and mouse primary cortical neurons (F) treated with 1 μg/ml of LRG1, DG-LRG1 or LRG1 variants (N79D, N186D, N269D, N325D). Scale bars, 200 μm for (E) and 100 μm for (F). βIII-tubulin-immunopositive neurite length was quantified in DRG explants and cortical neurons (200 cells/field) (right, mean±SEM (n=4)). The relative ratio of the LRG1-treated group was defined as 1.

*P<0.05; **P<0.01; ***P<0.001 (student's t test). N.S.: Not significant.

FIG. 10 shows sequence alignment of human LRG1 and structural comparison of LRG1 with other LRR family proteins.

FIG. 10A: results of sequence alignment of LRG1 derived from Homo sapiens (huLRG1, NP_443204.1), Macaca mulatta (maLRG1, EHH29497.1), Mus musculus (muLRG1, NP_084072.1), Rattus norvegicus (raLRG1, NP_001009717.1), Bos taurus (boLRG1, DAA2779 7. 1) and Canis lupus dingo (caLRG1, XP_025312813.1). The consensus sequence (LxxLxLxxNxL) and main conserved phenylalanines, disulfide bridges and glycosylated Asn residues are indicated on the bottom of the sequence alignment. Secondary structure elements are indicated at the top of the alignment (β-strands (arrows) and α-helices (cylinders)). Sequence alignments were obtained using T-Coffee (http://tcoffee.crg.cat) and the ESPript server (http://espript.ibcp.fr).

FIG. 10B: Structural comparison of the LRRNT and LRRCT domains of human LRG1 with other LRR proteins. Disulfide bridges are indicated by gray lines.

FIG. 11 illustrates the constructed LRG1-LPHN2 signaling pathway.

FIG. 11A: Results of Western blotting of immunoprecipitants with anti-LPHN2 antibody using antibodies specific for pY-LPHN2 and LPHN2. HUVECs were stimulated with LRG1 (1 μg/ml) or DG-LRG1 (1 μg/ml) for the indicated times under high glucose conditions.

FIG. 11B: Cignal Finder GPCR signaling 10-pathway reporter array analysis of HUVECs after treatment with LRG1 (1 μg/ml) or DG-LRG1 (1 μg/ml). The results of dual-luciferase assays are presented as normalized relative luminescence signals (means±SEM, n=3). The relative ratio of the untreated group was defined as 1.

FIG. 11C: Network model showing interactions between the proteins with increased phosphorylation in response to LRG1. Node color indicates the increase (red) in the phosphorylation level. The color bar indicates the gradient of the log 2-fold-change of phosphorylation levels by LRG1 with respect to those in untreated control conditions. Circled P on a node indicates phosphorylation of the corresponding protein. Protein interactions: activation (arrow); inhibition (solid lines interrupted by short lines); direct activation (solid arrows); indirect activation (dotted arrows); protein-protein interactions (gray lines); plasma membrane (green lines).

FIG. 11D: Western blot analysis of HUVECs stimulated with LRG1 (1 μg/ml) or DG-LRG1 (1 μg/ml) for the indicated times.

FIG. 11E:

Top image: BIII-tubulin staining of HUVECs tube formation (top), mouse DRG explants (middle) and mouse primary cortical neurons (bottom). HUVECs, mouse DRG explants and mouse primary cortical neurons were treated with DG-LRG1 (1 μg/ml), LY (10 μM), PP2 (10 μM), DG-LRG1 (1 μg/ml)+LY (10 μM), or DG-LRG1 (1 μg/ml)+PP2 (10 μM) under normal glucose conditions. Scale bars, 100 μm.

Bottom graph: quantification of master junctions, βIII-tubulin-immunopositive axon lengths in DRGs and the mean length of BIII-tubulin-immunopositive neurites at cortical neurons (n=4 fields, 200 cells/field) (means±SEM (n=4). The relative ratio of the non-treated group was defined as 1.

FIG. 11F: Results of Western blot of HUVECs stimulated with LRG1 or DG-LRG1 alone or in combination with TGF-β1.

FIGS. 11G and 11H: results of Western blots using antibodies specific for angiogenic factors (VEGF, angiopoietin-1, FGF2) (G) and neurotrophic factors (NGF, BDNF, NT-3) (H) in HUVECs (G) and mouse primary cortical neurons (H) stimulated with 1 μg/ml of LRG1 or DG-LRG1 (NGF, BDNF, NT-3) (H). In FIG. 11H, mouse primary cortical neurons were treated with shLPHN2 or shCon and then stimulated with 1 μg/ml of LRG1 or DG-LRG1.

In FIGS. 11A, 11B, and 11E, **P<0.01; ***P<0.001 (Student's t test). N.S.: not significant

FIG. 12 illustrates the LRG1/LPHN2-mediated signaling pathway.

FIG. 12A: altered protein and phosphorylation levels of the proteins after DG-LRG1 treatment. Colors indicate the increase (red) and decrease (blue) in the levels by DG-LRG1. Color bar represents the gradient of the log 2-fold-change of protein or phosphorylation levels by DG-LRG1 with respect to those in non-treated control conditions. Whether protein and phosphorylation levels are displayed is indicated by “Ab-” and “Phospho-” in the labels within parentheses, respectively. NF-κB and upstream phosphorylated proteins (LYN and AKT1) are denoted in red.

FIG. 12B: results of Western blot of HUVECs (left) or cortical neurons (right). After treatment with shLPHN2 or control shRNA (shCon), they were stimulated with 1 μg/ml of LRG1 or DG-LRG1 for the indicated times.

FIG. 12C: results of Western blot of HUVECs stimulated with 1 μg/ml of DG-LRG1 with or without pretreatment with PP2 (10 μM) or LY294002 (10 μM).

FIG. 12D: Western blot results of treatment of HUVECs with shLPHN2 or control shRNA (shCon) and then stimulation with 1 μg/ml LRG1 or DG-LRG1 in combination with TGF-1.

### BEST MODE

Unless defined otherwise, all technical and scientific terms used herein have the same meanings as appreciated by those skilled in the field to which the present invention pertains. In general, the nomenclature used herein is well-known in the art and is ordinarily used.

Unless otherwise indicated, nucleic acids and amino acids are recorded in orientations from left to right, from 5′ to 3′ and from N-terminus to C-terminus. Numerical ranges mentioned herein include the numbers defining the ranges and include each integer or non-integer fraction within the defined ranges.

Unless defined otherwise, all technical and scientific terms used herein have the same meanings as appreciated by those skilled in the field to which the present invention pertains. Although any methods and materials similar or equivalent to those described herein can be used to test the present invention, preferred materials and methods are described herein.

LRG1 is a glycoprotein present in human serum, the expression thereof is increased in various diseases, and the use thereof as a biomarker has been mainly reported. Recently, the effects of LRG1 on TGF-β-dependent angiogenesis and neurogenesis have been reported.

Based on the TGF-β/ENG-dependent angiogenesis and neurotization effects of the LRG1 glycoprotein, the present inventors found that the fusion protein of LRG1 and the Fc domain induces the regeneration of corpus cavernosum endothelial cells and nerve cells and thus is highly effective in increasing penile erection and regenerating blood vessels and nerves, and registered a patent associated with the use of the fusion protein for the treatment of erectile dysfunction, ischemic disease, and neurological diseases (Korea Patent Nos. 2162934 and 2002866).

However, it is difficult to fully explain the vascular and nerve regenerative effects of LRG1 by the weak binding affinity (˜2.9 μM) of LRG1 and ENG only with TGF-β-based mechanisms.

In one embodiment of the present invention, as a result of predicting the presence of vascular and neurogenic pathways in addition to the TGF-β/ENG-dependent pathway of LRG1 and conducting proteomics-based research to verify the prediction, LPHN2 was identified as a novel LRG1 receptor. Furthermore, it was found that angiogenesis and neurogenesis can be induced through the LPHN2 pathway even when treated with a TGF-β antibody under high-glucose conditions (e.g., diabetic subject) and LPHN2 is thus independent of the previously known LRG1/TGF-β pathway. However, it was found that natural LRG1 had no significant effect on blood vessels or neurons in normal mice.

In another embodiment of the present invention, functional activation mechanisms were identified under high-glucose conditions. Specifically, it was found that the LRG1 glycoprotein was deglycosylated under high-glucose conditions and that the deglycosylated LRG1 had a very high LPHN2 binding affinity compared to normal LRG1. Furthermore, it was found that deglycosylated LRG1 exhibits angiogenic and neurotrophic effects even under normal glucose conditions, which indicates that deglycosylation of LRG1 is a functional activation mechanism under high-glucose conditions for angiogenic and neurotrophic effects, and in another embodiment, angiogenesis and neurotization pathways of deglycosylated LRG1/LPHN2 were established.

In one aspect, the present invention is directed to a LRG1 glycoprotein (leucine rich α-2 glycoprotein) in which at least one glycosyl group is deglycosylated.

The LRG1 glycoprotein in which at least one glycosyl group is deglycosylated is highly effective in inducing angiogenesis, neurogenesis and/or neurotization, independent of the TGF-β pathway.

The LRG1 glycoprotein in which at least one glycosyl group is deglycosylated interacts with LPHN2 and induces downstream signaling thereof.

As used herein, the term “LRG1 (leucine-rich alpha-2-glycoprotein 1)” or “LRG1 glycoprotein” means a serum glycoprotein having leucine repeat units. The LRG1 glycoprotein includes LRG1 glycoproteins derived from various animals such as humans, apes, horses, pigs, rabbits, and mice, preferably mammals, and functionally identical proteins thereto. Preferably, the LRG1 glycoprotein may be derived from humans. In the present invention, LRG1 glycoprotein includes full-length glycoproteins and fragments of LRG1 glycoproteins that retain substantially identical functions and/or effects thereto.

In the present invention, the LRG1 glycoprotein may include an amino acid sequence represented by SEQ ID NO: 1.

The amino acid sequence represented by SEQ ID NO: 1 is human LRG1 protein and is used in the examples of the present invention. Human LRG1 (SEQ ID NO: 1) is known to include five glycosylation sites (T37: O-linked, others: N-linked) having glycosyl groups at residues T37, N79, N186, N269, and N325 (UniProt ID: P02750/A2GL_HUMAN; Reference Seq ID: NP_443204.1).

In addition, amino acids 1 to 35 of SEQ ID NO: 1 are signal peptides and amino acids 36 to 347 (SEQ ID NO: 2) correspond to active sequences.

Therefore, in the present invention, the LRG1 glycoprotein may include the amino acid sequence represented by SEQ ID NO: 2.

The glycosylation sites of SEQ ID NO: 1, residues T37, N79, N186, N269, and N325, respectively, correspond to residues T2, N44, N151, N234, and N290 of SEQ ID NO: 2.

The LRG1 glycoprotein according to the present invention is interpreted as including variants in which at least one amino acid residue is conservatively substituted at a specific residue position.

As used herein, the term “conservative substitution” refers to a modification of a LRG1 glycoprotein that includes substitution of one or more amino acids with other amino acids having similar biochemical properties that do not result in loss of the biological or biochemical function of the LRG1 glycoprotein.

The term “conservative amino acid substitution” refers to substitution of an amino acid residue with another amino acid residue having a similar side chain. Series of amino acid residues having similar side chains have been defined and are well known in the art to which the present invention pertains. These series include amino acids with basic side chains (e.g., lysine, arginine and histidine), amino acids with acidic side chains (e.g., aspartic acid and glutamic acid), amino acids with uncharged polar side chains (e.g., glycine, asparagine, glutamine, serine, threonine, tyrosine, and cysteine), amino acids with nonpolar side chains (e.g., alanine, valine, leucine, isoleucine, proline, phenylalanine, methionine, and tryptophan), amino acids with beta-branched side chains (e.g., threonine, valine, and isoleucine), and amino acids with aromatic side chains (e.g., tyrosine, phenylalanine, tryptophan, and histidine).

In addition, the LRG1 glycoprotein is interpreted to include human LRG1 glycoprotein including a sequence represented by SEQ ID NO: 1 or LRG1 glycoprotein including a sequence represented by SEQ ID NO: 2 and LRG1 glycoproteins or fragments thereof having substantially the same function and/or effect as those/that of the LRG1 glycoproteins according to the present invention, and having amino acid sequence homology of at least 50%, at least 60%, at least 70%, preferably at least 80% or at least 85%, more preferably at least 90% or at least 95%, and most preferably at least 99% to the LRG1 glycoprotein according to the present invention.

As used herein, the term “fragment” refers to a partial fragment, from which a parental protein is cleaved, and may be a fragment, from which the C′-terminus and/or the N′-terminus are cleaved. In the present invention, the fragment means a fragment having substantially the same function and/or effect as the deglycosylated LRG1 glycoprotein of the present invention. For example, the fragment may include a fragment in which a signal sequence is cleaved from a full-length protein.

In one embodiment of the present invention, it was found that LRG1 glycoprotein was deglycosylated under high-glucose conditions and it was demonstrated that deglycosylation of LRG1 is a functional activation mechanism for angiogenic and neurotrophic effects under high-glucose conditions. In particular, it was found that deglycosylation of the N-linked glycosyl group of LRG1 glycoprotein is essential to induce LPHN2 pathway-dependent angiogenesis and neurotization. Thereamong, deglycosylation of residue N325 is most essential.

Therefore, in the present invention, the LRG1 glycoprotein is characterized in that at least one of the glycosyl groups bound to amino acids selected from N79, N186, N269, and N325 in the amino acid sequence represented by SEQ ID NO: 1 is deglycosylated.

Therefore, in the present invention, the LRG1 glycoprotein is characterized in that it includes the sequence of SEQ ID NO: 2 and at least one of the glycosyl groups bound to amino acids selected from amino acid sequences N44, N151, N234, and N290 represented by SEQ ID NO: 2 is deglycosylated.

In the present invention, more preferably, the LRG1 glycoprotein is characterized in that the glycosyl group bound to amino acid N325 in the amino acid sequence represented by SEQ ID NO: 1 is deglycosylated.

In the present invention, more preferably, the LRG1 glycoprotein is characterized in that it includes SEQ ID NO: 2 and the glycosyl group bound to amino acid N290 in the amino acid sequence represented by SEQ ID NO: 2 is deglycosylated.

In the present invention, only the glycosyl group bound to amino acid N325 of SEQ ID NO: 1 may be deglycosylated.

In the present invention, the LRG1 glycoprotein may include a sequence represented by SEQ ID NO: 2 and only the glycosyl group bound to amino acid N290 of SEQ ID NO: 2 may be deglycosylated.

In the present invention, amino acid N325 of SEQ ID NO: 1 may be deglycosylated and glycosyl groups bound to one or more other amino acids may be deglycosylated.

In the present invention, the LRG1 glycoprotein may include a sequence represented by SEQ ID NO: 2 and amino acid N290 of SEQ ID NO: 2 as well as glycosyl groups bound to one or more other amino acids may be deglycosylated.

In the present invention, when the LRG1 glycoprotein has an amino acid sequence different from the sequence of SEQ ID NO: 1 or SEQ ID NO: 2, for example, an amino acid sequence derived from another organism, those skilled in the art can easily derive amino acids corresponding to the glycosylation site, through sequence alignment and analysis, or the like. In this case, it is obvious that the deglycosylated LRG1 glycoprotein of the present invention may be characterized in that the glycosyl group of the amino acid at the site corresponding to the glycosylation site is deglycosylated.

In the present invention, the LRG1 glycoprotein may be bound to latrophilin-2 (LPHN2). As can be seen from one embodiment of the present invention, the deglycosylated LRG1 glycoprotein of the present invention may have a KD of 500 nM or less, preferably 480 nM or less, and most preferably 450 nM or less.

In the present invention, the deglycosylated LRG1 glycoprotein of the present invention has an LPHN2-expressing cell surface binding rate of 1.3 or more times, preferably 1.5 or more times, and most preferably 2 or more times normal LRG1 protein.

The deglycosylated LRG1 glycoprotein of the present invention may induce phosphorylation of at least one of Lyn, AKT, and NF-κB p65.

The deglycosylated LRG1 glycoprotein of the present invention may improve the expression of neurotrophic factors such as NGF, BDNF, and NTR3 in neurons.

As in one embodiment of the present invention, the deglycosylated LRG1 glycoprotein of the present invention may be prepared by treating an intact LRG1 glycoprotein with a deglycosylation enzyme such as PNGase F to prepare LRG1 glycoprotein, the glycosyl group of which is deglycosylated and then by preparing a variant including substitution of an amino acid at the glycosylation site for deglycosylation at a more specific glycosylation site.

In one embodiment of the present invention, deglycosylated LRG1 was prepared using two methods. In order to verify the importance of deglycosylation of LRG1, the deglycosylated LRG1 was prepared using PNGase F, which randomly degrades the N-linked glycan of LRG1 (Example 5), and LRG1 variants in which each of N79, N186, N269, and N325 were substituted with aspartic acid (D) were prepared and used, in order to prepare an LRG1 glycoprotein in which a glycosyl group is not linked to each glycosylation site.

In another aspect, the present invention is directed to an LRG1 glycoprotein (leucine rich α-2 glycoprotein) variant including a variation at one or more glycosylation sites.

As used herein, the term “glycosylation” refers to the most common form of modification of protein such as serine or asparagine, and refers to a process in which carbohydrate glycan binds to an amino acid residue, for example, oxygen in serine or nitrogen in asparagine. The glycosylation may affect various properties, such as secondary and tertiary protein structures, intercellular signaling, biological activity, and stability.

In the present invention, the glycosylation may be N-glycosylation (N-(linked) glycosylation), O-glycosylation (O-(linked) glycosylation), phosphoserine glycosylation, C-mannosylation, or the like, preferably N-glycosylation or O-glycosylation.

As used herein, the term “glycosylation site” refers to an amino acid in LRG1 glycoprotein, to which a glycosyl group binds, or an amino acid adjacent thereto affecting the binding of the glycosyl group. T73, N79, N186, N269, and N325 are known as glycosylation sites in human LRG1 (SEQ ID NO: 1), T73 is an O-linked glycosyl group, and the remaining four amino acids (N79, N186, N269, and N325) have N-linked glycosyl groups. LRG1 glycoproteins having a sequence other than the sequence of SEQ ID NO: 1 (e.g., LRG1 glycoprotein derived from other animals) may have different glycosylation sites. However, as can be seen from one embodiment of the present invention, the sites corresponding to N269 and Asn325 of SEQ ID NO: 1 in the glycosylation sites may be highly conserved.

In the present invention, the LRG1 glycoprotein variant may not include a glycosyl group at the varied glycosylation site.

In the present invention, the LRG1 glycoprotein variant includes a variation of LRG1 glycoprotein including the amino acid sequence of SEQ ID NO: 2.

In the present invention, the LRG1 glycoprotein variant includes a variation of LRG1 glycoprotein including the amino acid sequence of SEQ ID NO: 1.

In the present invention, the LRG1 glycoprotein variant includes variation in at least one amino acid selected from N79, N186, N269, and N325 of SEQ ID NO: 1.

In the present invention, the LRG1 glycoprotein variant includes an amino acid sequence including a variation in at least one amino acid selected from N44, N151, N234, and N290 of SEQ ID NO: 2.

In the present invention, most preferably, the LRG1 glycoprotein variant includes a variation in amino acid N325 of SEQ ID NO: 1.

In the present invention, the LRG1 glycoprotein variant includes an amino acid sequence including a variation in amino acid N290 of SEQ ID NO: 2.

In the present invention, the LRG1 glycoprotein variant includes a variation in only amino acid N325 of SEQ ID NO: 1.

In the present invention, the LRG1 glycoprotein variant includes a variation in amino acid N325 of SEQ ID NO: 1 and a variation in another amino acid.

In the present invention, the LRG1 glycoprotein variant includes a sequence of SEQ ID NO: 2 including a variation in only amino acid N290.

In the present invention, the LRG1 glycoprotein variant includes a sequence of SEQ ID NO: 2 including a variation in amino acid N290 and a variation in another amino acid.

As used herein, the term “variant” is interpreted as including variations of one or more amino acid residues, preferably, substitutions, deletions and insertions of one or more amino acid residues, more preferably, substitutions of one or more amino acid residues, and deletions of one or more N-terminal and/or C-terminal amino acid residues in the amino acid sequence of the reference sequence (e.g., normal LRG1 glycoprotein sequence, SEQ ID NO: 1). In one embodiment of the present invention, the variation has a configuration in which an amino acid in the glycosylation site of the LRG 1 glycoprotein is substituted with aspartic acid, but is not limited thereto.

In the present invention, the variation may be substitution with an amino acid.

In the present invention, the amino acid may be substituted with aspartic acid.

In the present invention, preferably, the LRG1 glycoprotein variant may include one or more amino acids selected from N79, N186, N269, and N325 of SEQ ID NO: 1 with other amino acids.

In the present invention, preferably, the LRG1 glycoprotein variant may include a sequence in which one or more amino acids selected from N44, N151, N234, and N290 of SEQ ID NO: 2 are substituted with other amino acids.

In the present invention, more preferably, the LRG1 glycoprotein variant may include substitution of amino acid N325 of SEQ ID NO: 1 with another amino acid, and most preferably, substitution of amino acid N325 with aspartic acid (D).

In the present invention, more preferably, the LRG1 glycoprotein variant may include a sequence in which N290 amino acid of SEQ ID NO: 2 is substituted with another amino acid, and most preferably, a sequence in which N290 amino acid of SEQ ID NO: 2 is substituted with aspartic acid (D).

In the present invention, the LRG1 glycoprotein variant may include substitution of one or more amino acid residues selected from the group consisting of N79D, N186D, N269D, and N325D in SEQ ID NO: 1.

In the present invention, preferably, the LRG1 glycoprotein variant may include a substitution of N325D in SEQ ID NO: 1.

In the present invention, the LRG1 glycoprotein variant may include a sequence including substitution of one or more amino acid residues selected from the group consisting of N44D, N151D, N234D, and N290D in SEQ ID NO: 2.

In the present invention, preferably, the LRG1 glycoprotein variant may include a substitution of N325D in SEQ ID NO: 1.

In the present invention, preferably, the LRG1 glycoprotein variant may include a sequence including a substitution of N290D in SEQ ID NO: 2.

In the present invention, an expression described by a one-letter amino acid residue code together with numbers, such as “N325”, means an amino acid residue and type at the n^{th} position in each amino acid sequence.

For example, “N325” means that the amino acid residue at position 325 in the amino acid sequence of SEQ ID NO: 1 is asparagine. In “N325D”, the name of the amino acid residue after the number means amino acid substitution and the “N325D” means that asparagine (Asn, N) at position 325 of SEQ ID NO: 1 is substituted with aspartic acid (Asp, D).

In the present invention, when the LRG1 glycoprotein variant has an amino acid sequence different from the sequence of SEQ ID NO: 1, for example, the LRG1 glycoprotein variant is a variant based on the LRG1 glycoprotein derived from another organism, those skilled in the art can easily derive amino acids corresponding to the glycosylation site through sequence alignment and analysis, or the like. In this case, it is obvious that the deglycosylated LRG1 variant of the present invention includes variation in the glycosyl glycosylation corresponding to the glycosylation site described above.

In the present invention, the LRG1 glycoprotein variant may bind to latrophilin-2 (LPHN2). As can be seen from one embodiment of the present invention, the deglycosylated LRG1 glycoprotein of the present invention may have a KD of 500 nM or less, preferably 480 nM or less, and most preferably 450 nM or less.

In the present invention, the deglycosylated LRG1 variant of the present invention has an LPHN2-expressing cell surface binding rate of 1.3 or more times, preferably 1.5 or more times, and most preferably 2 or more times normal LRG1 protein.

The deglycosylated LRG1 variant of the present invention may induce phosphorylation of at least one of Lyn, AKT, and NF-κB p65.

The deglycosylated LRG1 variant of the present invention may improve the expression of neurotrophic factors such as NGF, BDNF, and NTR3.

In the present invention, the LRG1 glycoprotein variant is interpreted as including a fragment thereof.

The LRG1 glycoprotein variant of the present invention strongly binds to and interacts with LPHN2, and induces growth of blood vessels and/or nerve cells, angiogenesis, and nerve regeneration through a signaling pathway involving Lyn, AKT, and/or NF-κB p65.

In another aspect, the present invention is directed to a nucleic acid encoding the LRG1 glycoprotein variant according to the present invention.

The nucleic acids, as used herein, may be present in cells, in a cell lysate, or in a partially purified or substantially pure form. “Isolated” or “substantially pure”, when referring to nucleic acids, refers to those that have been purified and thus separated from other cellular components or other contaminants, e.g., other cellular nucleic acids or proteins, by standard techniques, including alkaline/SDS treatment, CsCl banding, column chromatography, agarose gel electrophoresis, and other techniques well known in the art. The nucleic acids of the present invention may be DNA or RNA.

In still another aspect, the present invention is directed to a vector including the nucleic acid.

For expression of the LRG1 glycoprotein variant according to the present invention, a DNA encoding the LRG1 glycoprotein variant can be obtained by standard molecular biology techniques (e.g., PCR amplification or cDNA cloning using a hybridoma that expresses the LRG1 glycoprotein variant), and the DNA can be inserted into an expression vector such that it is “operatively linked” to transcriptional and translational control sequences.

The “vector” means a DNA product containing a DNA sequence operably linked to a suitable regulatory sequence capable of expressing the DNA in a suitable host. Vectors may be plasmids, phage particles or simply potential genomic inserts. When transformed into a suitable host, vectors may be replicated or perform functions independent of the host genomes, or some thereof may be integrated with the genomes. A plasmid is currently the most commonly used form of vector, and thus the terms “plasmid” and “vector” are often used interchangeably. However, the present invention encompasses other forms of vectors that are known in the art or have the same functions as those known in the art. Protein expression vectors used in E. coli include: the pET family vectors from Novagen, Inc (USA); the pBAD family vectors from Invitrogen Corp. (USA); PHCE or pCOLD vectors from Takara Bio Inc. (Japan); and pACE family vectors from GenoFocus Inc. (South Korea). In Bacillus subtilis, a gene of interest can be inserted into a specific part of the genome to realize protein expression, or a pHT-family vector of MoBiTech (Germany) can be used. Even in fungi and yeast, protein expression is possible using genome insertion or self-replicating vectors. A plant protein expression vector using a T-DNA system such as Agrobacterium tumefaciens or Agrobacterium rhizogenes can be used. Typical expression vectors for expression in mammalian cell cultures are based on, for example, pRK5 (EP 307,247), pSV16B (WO 91/08291), and pVL1392 (Pharmingen).

As used herein, the term “expression control sequence” means a DNA sequence essential for the expression of a coding sequence operably linked to a particular host organism. Such a control sequence includes promoters for conducting transcription, operator sequences for controlling such transcription, sequences for encoding suitable mRNA ribosome-binding sites, and sequences for controlling the termination of transcription and translation. For example, control sequences suitable for prokaryotes include promoters, optionally operator sequences, and ribosome-binding sites. Control sequences suitable for eukaryotic cells include promoters, polyadenylation signals, and enhancers. The factor that has the greatest impact on the expression level of a gene in a plasmid is the promoter. SRα promoters, cytomegalovirus-derived promoters and the like are preferably used as promoters for high expression.

Any of a wide variety of expression control sequences may be used for the vector in order to express the DNA sequences of the present invention. Useful expression control sequences include, for example, early and late promoters of SV40 or adenovirus, the lac system, the trp system, the TAC or TRC system, T3 and T7 promoters, the major operator and promoter regions of phage lambda, control regions of fd code proteins, promoters of 3-phosphoglycerate kinase or other glycol lyases, promoters of the phosphatase, such as Pho5, promoters of yeast alpha-mating systems, and other sequences having configurations and induction activity known to control gene expression of prokaryotic or eukaryotic cells or viruses and various combinations thereof. The T7 RNA polymerase promoter Φ10 may be useful for expressing proteins in E. coli.

Those skilled in the art will recognize that the design of an expression vector may be changed by selecting various regulatory sequences in consideration of factors such as the selection of host cells to be transformed, the expression level of proteins, and the like.

When a nucleic acid sequence is aligned with another nucleic acid sequence based on a functional relationship, it is “operably linked” thereto. This may be gene (s) and control sequence(s) linked in such a way so as to enable gene expression when a suitable molecule (e.g., a transcriptional activator protein) is linked to the control sequence(s). For example, DNA for a pre-sequence or secretory leader is operably linked to DNA for a polypeptide when expressed as a pre-protein involved in the secretion of the polypeptide; a promoter or enhancer is operably linked to a coding sequence when it affects the transcription of the sequence; or a ribosome-binding site is operably linked to a coding sequence when it affects the transcription of the sequence; or the ribosome-binding site is operably linked to a coding sequence when positioned to facilitate translation. Generally, the term “operably linked” means that the linked DNA sequence is in contact therewith, and that a secretory leader is in contact therewith and is present in the reading frame. However, the enhancer need not be in contact therewith. The linkage of these sequences is carried out by ligation (linkage) at convenient restriction enzyme sites. When no such site exists, a synthetic oligonucleotide adapter or a linker according to a conventional method is used.

As used herein, the term “expression vector” commonly refers to a recombinant carrier into which a fragment of heterologous DNA is inserted, and generally means a fragment of double-stranded DNA. Herein, “heterologous DNA” means xenogenous DNA that is not naturally found in the host cell. Once an expression vector is present in a host cell, it can replicate independently of the host chromosomal DNA, and several copies of the vector and inserted (heterologous) DNA thereof can be produced.

As is well known in the art, in order to increase the expression level of a transfected gene in a recombinant cell, the gene should be operably linked to a transcriptional or translational expression control sequence that functions in the selected expression host. Preferably, the expression control sequence and the corresponding gene are included in a single expression vector containing both a bacterial selection marker and a replication origin. When the expression host is a eukaryotic cell, the expression vector should further include a useful expression marker in the eukaryotic expression host.

In yet another aspect, the present invention is directed to a host cell including the nucleic acid or the vector.

As used herein, the term “host cell” refers to a cell for expression into which a nucleic acid encoding a LRG1 glycoprotein variant or a vector containing the nucleic acid has been introduced to produce the LRG1 glycoprotein variant. The host cell may be used without limitation as long as it is a cell capable of expressing a LRG1 glycoprotein variant, and may preferably be a eukaryotic cell, more preferably yeast, an insect cell, or an animal cell, and most preferably an animal cell. For example, a CHO cell line or a HEK cell line mainly used for expression of recombinant proteins may be used, and in one embodiment of the present invention, Freestyle 293-F, which is a HEK cell line, was used, but the invention is not limited thereto.

In the present invention, as the host cells for expressing the recombinant protein, prokaryotic cells such as Escherichia coli and Bacillus subtilis, which enable cell culture at a high concentration within a short time, can be easily genetically manipulated, and the genetic and physiological characteristics of which are well known, have been widely used. However, in order to solve problems such as post-translational modification of proteins, secretion process and three-dimensional structure of the active form, and the active state of the protein, single-celled eukaryotic yeasts (such as Pichia pastoris, Saccharomyces cerevisiae, and Hansenula polymorpha), filamentous fungi, insect cells, plant cells, and cells of higher organisms such as mammals have been recently used as host cells for the production of recombinant proteins. Thus, the use of other host cells as well as Bacillus described in the examples will be readily evident to those skilled in the art.

A wide variety of expression host/vector combinations can be used to express the LRG1 glycoprotein variants of the present invention. Suitable expression vectors for eukaryotic hosts include, for example, expression control sequences derived from SV40, cow papillomavirus, adenovirus, adeno-associated virus, cytomegalovirus and retrovirus. Expression vectors that can be used for bacterial hosts include bacterial plasmids that may be exemplified by those obtained from E. coli, such as pBlueScript, pGEX2T, pUC vectors, col E1, pCR1, pBR322, pMB9 and derivatives thereof, plasmids having a wide host range such as RP4, phage DNA that may be exemplified by a wide variety of phage lambda derivatives such as λgt10, λgt11 and NM989, and other DNA phages such as M13 and filamentous single-stranded DNA phages. Expression vectors useful for yeast cells include 2p plasmids and derivatives thereof. A vector useful for insect cells is pVL 941.

In one embodiment of the present invention, a host cell/vector combination of Freestyle 293-F cell line, as a HEK cell line and a ppcDNA3.1 vector was used to produce the LRG1 glycoprotein variants, but is not limited thereto.

The vector may be introduced into a host cell through a method such as transformation or transfection. As used herein, the term “transformation” means introducing DNA into a host and making the DNA replicable using an extrachromosomal factor or chromosomal integration. As used herein, the term “transfection” means that an expression vector is accommodated by the host cell, regardless of whether or not any coding sequence is actually expressed. In order to introduce the vector, various techniques commonly used to introduce foreign nucleic acids (DNA or RNA) into prokaryotic or eukaryotic host cells, for example, electrophoresis, calcium phosphate precipitation, DEAE-dextran transfection or lipofection may be used, but are not limited thereto.

It should be understood that not all vectors function identically in expressing the DNA sequences of the present invention. Likewise, not all hosts function identically for the same expression system. However, those skilled in the art will be able to make appropriate selections from among a variety of vectors, expression control sequences and hosts without excessive burden of experimentation and without departing from the scope of the present invention. For example, selection of a vector should be carried out in consideration of a host because the vector should be replicated therein. The number of replications of the vector, the ability to control the number of replications, and the expression of other proteins encoded by the corresponding vector, such as the expression of antibiotic markers, should also be considered. In selecting the expression control sequence, a number of factors should be considered. For example, the relative strength of the sequence, controllability, and compatibility with the DNA sequences of the present invention should be considered, particularly in relation to possible secondary structures. A single-cell host may be selected in consideration of factors such as the selected vector, the toxicity of the product encoded by the DNA sequence of the present invention, secretion characteristics, the ability to accurately fold proteins, culture and fermentation factors, and ease of purification of the product encoded by the DNA sequence according to the present invention. Within the scope of these factors, those skilled in the art can select various vector/expression control sequence/host combinations capable of expressing the DNA sequences of the present invention in fermentation or large animal cultures. As a screening method for cloning cDNA of proteins through expression cloning, a binding method, a panning method, a film emulsion method or the like can be applied.

In the present invention, the nucleic acid encoding the LRG1 glycoprotein variant may be directly introduced into the genome of a host cell and present as a factor on a chromosome. It will be apparent to those skilled in the art to which the present invention pertains that even if the gene is inserted into the genomic chromosome of the host cell, it will have the same effect as when the recombinant vector is introduced into the host cell.

In another aspect, the present invention is directed to a method of producing an LRG1 glycoprotein variant including culturing the host cell.

When a recombinant expression vector capable of expressing the LRG1 glycoprotein variant is introduced into mammalian host cells, the LRG1 glycoprotein variant or a variant thereof can be produced by culturing the host cell for a period of time such that the LRG1 glycoprotein variant is expressed in the host cells, preferably a period of time such that the LRG1 glycoprotein variant is secreted into the medium during culture of the host cells.

In some cases, the expressed LRG1 glycoprotein variant may be isolated and purified from the host cells. Isolation or purification of the LRG1 glycoprotein variant can be performed by conventional isolation/purification methods (e.g., chromatography) that are used for proteins. The chromatography may include a combination of one or more selected from affinity chromatography, ion exchange chromatography, and hydrophobic chromatography, but is not limited thereto. In addition to the chromatography, a combination of filtration, ultrafiltration, salting out, dialysis, and the like may be used.

The present inventors registered a patent associated with the use of the LRG1-Fc fusion protein, in which LRG1 is fused with a Fc domain, for the treatment of erectile dysfunction, ischemic disease, and neurological diseases (Korea Patent Nos. 2162934 and 2002866).

In one embodiment of the present invention, an LRG1-Fc fusion protein was also recombined and the effects of inducing angiogenesis and neurogenesis are effectively achieved through LPHN2 even in the presence of a TGF-β antibody under high-glucose conditions in which deglycosylation of LRG1 glycoprotein occurs.

In another aspect, the present invention is directed to a fusion protein in which an Fc domain is fused to the deglycosylated LRG1 glycoprotein or the LRG1 glycoprotein variant.

As used herein, the term “Fc domain” means an Fc domain of Ig (immunoglobulin). The Fc domain of the Ig means a heavy-chain constant region 2 (CH2) and a heavy-chain constant region 3 (CH3), excluding the heavy- and light-chain variable regions, a heavy-chain constant region 1 (CH1) and a light-chain constant region (CL1) of the Ig, and a hinge domain may be included in the heavy chain constant region. The Fc domain of the Ig may be an extended Fc region including a part or all of the heavy-chain constant region 1 (CH1) and/or light-chain constant region 1 (CL1), excluding only the heavy- and light-chain variable regions of Ig, as long as the Fc domain of the Ig has an effect substantially similar or superior to that of the wild-type. In addition, the Fc domain may be a region from which a part of very long amino acid sequence corresponding to CH2 and/or CH3 is removed. In addition, the domain of Ig may include a variety of derivatives such as a derivative in which a site where a disulfide bond is formed is removed, a derivative in which one or more amino acids are removed from the N-terminus of wild-type Fc, or a derivative in which a methionine residue is added to the N-terminus of wild-type Fc.

In the present invention, Ig includes IgG, IgA, IgD, IgE, and IgM, preferably, IgG. The IgG may originate from humans or animals such as cattle, goats, pigs, mice, rabbits, hamsters, rats or guinea pigs, preferably humans. The IgG includes IgG1, IgG2, IgG3 or IgG4, preferably IgG1.

In the present invention, the Fc domain may be fused to the N′-terminus and/or C′-terminus of the deglycosylated LRG1 glycoprotein or LRG1 glycoprotein variant.

The fusion protein of the present invention may have a configuration in which leucine-rich alpha-2-glycoprotein 1 (LRG1) glycoprotein is bound to the Fc domain via a linker.

As used herein, the term “linker” basically refers to a means for binding two different fusion partners (e.g., biological polymers, etc.) through hydrogen bonds, electrostatic interactions, van der Waals forces, disulfide bonds, salt bridges, hydrophobic interactions, covalent bonds, and the like. In the present invention, the linker is preferably a peptide linker.

As used herein, the term “peptide linker” refers to a peptide having an arbitrary amino acid sequence and may be a peptide linker that includes small-sized amino acids having no functional group, preferably one or more amino acids selected from the group consisting of alanine, glycine and combinations thereof. The peptide linker may include a number of amino acids capable of providing flexibility to maintain appropriate culturability, for example, several to several tens of amino acids, while not interfering with the binding ability with the LRG1 glycoprotein and the Fc domain.

In the present invention, the Fc domain binds to a neonatal Fc-receptor (FcRN) on the surface of vascular endothelial cells and binds to the FcRN at a low pH and unbinds therefrom at neutral pH. Due to these characteristics, the fusion protein including the Fc domain is endocytosed into vascular endothelial cells, exocytosed again and then discharged into the blood. For this reason, the half-life of the fusion protein in blood is significantly increased. In addition, the fusion protein containing the Fc domain is more advantageous for improving solubility and safety by fusion with the Fc domain, and for reducing drug preparation costs due to simplified purification and separation, compared to when only a LRG1 glycoprotein protein is used.

The deglycosylated LRG1 glycoprotein, LRG1 glycoprotein variant and/or fragment thereof according to the present invention strongly binds to and interacts with LPHN2, and induces the growth of blood vessels and/or nerve cells, angiogenesis, and neurotization by direct signaling pathways including Lyn, AKT, and/or NF-κB p65, and enhancement of the expression of neurotrophic factors including NGF, BDNF, and NT3.

In one embodiment of the present invention, a deglycosylated LRG1 glycoprotein is prepared by treatment with the PNGaseF of the present invention, an LRG1 glycoprotein variant including a variation at the glycosylation site (hereinafter collectively referred to as DG-LRG1) is prepared, and the DG-LRG1 remarkably induces vascular endothelial and nerve growth in a TGF-β-independent manner through direct effect of the LPHN2 pathway and indirect effects of enhancing the expression of neurotrophic factors, and has effects of improving angiogenesis and regeneration of peripheral nerves, and alleviating erectile dysfunction in diabetic model mice with complications such as ischemic and neurological diseases.

In another aspect, the present invention is directed to a composition for inducing angiogenesis, neurogenesis and neurotization containing the LRG1 glycoprotein, the LRG1 glycoprotein variant, or the fusion protein.

In another aspect, the present invention is directed to a composition for preventing or treating an ischemic disease containing the LRG1 glycoprotein, the LRG1 glycoprotein variant, or the fusion protein.

In another aspect, the present invention is directed to a composition for preventing or treating a peripheral nerve disease containing the LRG1 glycoprotein, the LRG1 glycoprotein variant, or the fusion protein.

In another aspect, the present invention is directed to a composition for preventing or treating erectile dysfunction containing the LRG1 glycoprotein, the LRG1 glycoprotein variant, or the fusion protein.

In another aspect, the present invention is directed to is a composition for preventing or treating a neurodegenerative disease containing the LRG1 glycoprotein, the LRG1 glycoprotein variant, or the fusion protein.

As used herein, the term “angiogenesis” refers to a general term for a series of processes in which new blood vessels are formed from existing blood vessels. The angiogenesis may include formation of new blood vessels through division of endothelial cells or the like. In the present invention, the angiogenesis may include intussusceptive angiogenesis and sprouting angiogenesis.

The deglycosylated LRG1 glycoprotein, LRG1 glycoprotein variant, or fusion protein according to the present invention may induce angiogenesis by activation of a signaling pathway based on direct binding to LPHN2.

As used herein, the term “neurogenesis (nerve growth)” refers to the growth and expansion of nerve cells and networks, including neurite outgrowth and axonal sprouting. In the present invention, neurogenesis (nerve growth) may be used as “neurotization (nerve regeneration)” in terms of repair of damaged nerve cells.

As used herein, the term “neurotization (nerve regeneration)” means the recovery of damaged nerve cells.

The deglycosylated LRG1 glycoprotein, LRG1 glycoprotein variant, or fusion protein of the present invention may induce nerve growth through activation of signaling pathways (direct effect) based on direct binding to LPHN2 and enhancement of expression of neurotrophic factors.

As used herein, the term “ischemic disease” refers to a disease with reduced blood supply to an organ, tissue or site and examples of the ischemic disease include myocardial infarction, cerebral infarction, ischemic acute renal failure, ischemic acute liver failure, diabetic foot ulcer, diabetic nephropathy, ischemic colitis, myocardial hypertrophy, and ischemic disease or organ tissue damage due to surgical side effects, which are classified depending on the related organ or cause, but are not limited thereto. Ischemic diseases caused by surgical side effects include, but are not limited to, ischemic heart failure, ischemic renal failure, ischemic liver failure, or ischemic stroke. The organ tissue damage includes damage caused by long-term surgery or transplant or traumatic amputated limb reattachment with reperfusion after ischemia. The organ may include, but is not limited to, kidney, liver, pancreas, lung, heart or the like.

As used herein, the term “peripheral nervous disease” refers to a disease caused by damage or death of peripheral nerves due to various causes. The peripheral nervous system disease includes, but is not limited to, peripheral neuropathy, diabetic neuropathy, trophic neuropathy, trigeminal neuralgia, sciatica, carpal tunnel syndrome, facial paralysis, (post-surgical/traumatic) peripheral nerve injury, myasthenia gravis, Guillain-Barre syndrome, neurogenic tics, and the like.

As used herein, the term “erectile dysfunction” refers to the inability to achieve or maintain an erection sufficient for sexual intercourse and is generally defined as a state in which the inability lasts for more than 3 months. The erectile dysfunction is interpretated as having a broad meaning including psychogenic erectile dysfunction caused by psychological factors, vasculogenic erectile dysfunction, neurogenic erectile dysfunction, endocrine erectile dysfunction, erectile dysfunction due to metabolic syndrome, erectile dysfunction caused by drug side effects, and the like. The composition of the present invention can be used for the prevention and/or treatment of erectile dysfunction caused by all of the causes, preferably for vasculogenic erectile dysfunction and/or neurogenic erectile dysfunction, but is not limited thereto.

As used herein, the term “degenerative neurological disease” or “neurodegenerative disease” refers to a disease with a degenerative condition of nerve cells in the central nervous system, particularly the brain, and various symptoms due to the loss of the intrinsic function of the degenerative area. Examples of the degenerative neurological disease include Alzheimer's disease, Parkinson's disease, amyotrophic lateral sclerosis, Huntington's chorea, Creutzfeldt-Jakob disease, and degenerative brain diseases, but are not limited thereto.

As used herein, the term “prevention” refers to any action that inhibits the onset of a target disease or delays the progression of the disease through administration of the composition of the present invention.

As used herein, the term “treatment” refers to inhibition of the onset of cancer, or alleviation or elimination of symptoms of a target disease through administration of the pharmaceutical composition of the present invention.

In the present invention, the composition may be a pharmaceutical composition.

The pharmaceutical composition may further contain a pharmaceutically acceptable carrier, excipient or diluent.

Examples of the carrier, excipient and diluent that may be contained in the pharmaceutical composition may include lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate, mineral oil and the like. Upon formulation of the composition, typically used diluents or excipients, such as fillers, extenders, binders, wetting agents, disintegrants or detergents, may be used.

The pharmaceutical composition according to the present invention can be formulated and used in various forms according to a conventional method. Suitable formulations include oral formulations such as tablets, pills, powders, granules, dragées, hard or soft capsules, solutions, suspensions, emulsions, injections and aerosols, external preparations, suppositories, sterile injectable solutions, and the like, but are not limited thereto.

The pharmaceutical composition according to the present invention can be prepared into a suitable formulation using a pharmaceutically inactive organic or inorganic carrier. That is, when the formulation is a tablet, a coated tablet, a dragée or a hard capsule, it may contain lactose, sucrose, starch or a derivative thereof, talc, calcium carbonate, gelatin, stearic acid, or a salt thereof. In addition, when the formulation is a soft capsule, it may contain a vegetable oil, wax, fat, or semi-solid or liquid polyol. In addition, when the formulation is in the form of a solution or syrup, it may contain water, polyol, glycerol, vegetable oil, or the like.

The pharmaceutical composition according to the present invention may further contain a preservative, a stabilizer, a wetting agent, an emulsifier, a solubilizing agent, a flavoring agent, a colorant, an osmotic pressure regulator, an antioxidant or the like, in addition to the above carrier.

The pharmaceutical composition according to the present invention may be administered in a pharmaceutically effective amount, and the term “pharmaceutically effective amount” refers to an amount sufficient for treating a disease at a reasonable benefit/risk ratio applicable to all medical treatments, and the effective dosage level may be determined depending on a variety of factors including the type of the disease of the patient, the severity of the disease, the activity of the drug, the sensitivity of the patient to the drug, the administration time, the administration route, the excretion rate, the treatment period, drugs used concurrently therewith, and other factors well-known in the field of pharmaceuticals. The pharmaceutical composition of the present invention may be administered as a single therapeutic agent or in combination with other therapeutic agents, may be administered sequentially or simultaneously with a conventional therapeutic agent, and may be administered in one or multiple doses. Taking into consideration these factors, it is important to administer the minimum amount sufficient to achieve maximum efficacy without side effects, and the amount can be easily determined by those skilled in the art.

The pharmaceutical composition may be administered orally or parenterally. Parenteral administration is carried out by intravenous injection, subcutaneous injection, intramuscular injection, intraperitoneal injection, endothelial administration, topical administration, intranasal administration, intrapulmonary administration, rectal administration, and the like. For oral administration, the active ingredient in the oral composition needs to be formulated into a coated dosage form or into a dosage form that can protect the active ingredient from disintegrating in the stomach, considering that peptides and proteins are digested in the stomach. Alternatively, the present composition may be administered via any device by which the active ingredient can move to the target cell of interest.

The administration method of the pharmaceutical composition according to the present invention can be easily selected according to the formation and can be administered orally or parenterally. The dosage may vary depending on the age, gender, weight, severity of symptoms of the patient, and route of administration.

The composition according to the present invention may be used in combination with another composition or therapy for treating ischemic diseases, peripheral nervous diseases, erectile dysfunction or neurodegenerative diseases.

In another aspect, the present invention is directed to a method for inducing angiogenesis, neurogenesis and/or neurotization including administering the LRG1 glycoprotein, the LRG1 glycoprotein variant, or the fusion protein to a subject.

In another aspect, the present invention is directed to a method for treating ischemic diseases, peripheral nerve diseases, erectile dysfunction and/or neurodegenerative diseases including administering the LRG1 glycoprotein, LRG1 glycoprotein variant, or fusion protein to a subject.

In another aspect, the present invention is directed to the use of the LRG1 glycoprotein, the LRG1 glycoprotein variant, or the fusion protein for inducing angiogenesis, neurogenesis and/or neurotization.

In another aspect, the present invention is directed to the use of the LRG1 glycoprotein, the LRG1 glycoprotein variant, or the fusion protein for treating ischemic diseases, peripheral nerve diseases, erectile dysfunction and/or neurodegenerative diseases.

In another aspect, the present invention is directed to the use of the LRG1 glycoprotein, the LRG1 glycoprotein variant, or the fusion protein for the preparation of a composition for inducing angiogenesis, neurogenesis and/or neurotization.

In another aspect, the present invention is directed to the use of the LRG1 glycoprotein, the LRG1 glycoprotein variant, or the fusion protein for the preparation of a composition for treating ischemic diseases, peripheral nerve diseases, erectile dysfunction and/or neurodegenerative diseases.

### EXAMPLE

Hereinafter, the present invention will be described in more detail with reference to examples. However, it will be obvious to those skilled in the art that these examples are provided only for illustration of the present invention, and should not be construed as limiting the scope of the present invention.

### Example 1: Materials and Methods

### Example 1-1: Cell Culture

Human umbilical vein endothelial cells (HUVECs; Cat #CC-2519, Lonza) and human embryonic kidney 293T cells (HEK293T; Cat #CRL-3216, ATCC) were certified in accordance with ATCC guidelines and used within 6 months of receipt.

The HUVECs were cultured in EGM-2 (Cat #CC-3156, Lonza) supplemented with 2 mM L-glutamine (Cat #25030081, Gibco), 100 U/ml of penicillin and 100 μg/ml of streptomycin (Cat #15140122, Gibco) on gelatin (Cat #G1890, Sigma-Aldrich; 0.1% in DDW) pre-coated plate.

HEK293T cells were cultured in DMEM (Cat #41965039, Gibco) supplemented with 10% fetal bovine serum (FBS; Cat #10270106, Gibco) and 100 μg/ml antibiotic-antimycotic (Cat #15240062, Gibco). The cells were incubated at 37° C. under 5% humidity and CO₂ conditions. The cells with a passage between 2 and 7 were used in all examples and all experiments were performed in accordance with the guidelines of the organization.

Primary mouse spongiform endothelial cells (MCEC) were prepared and kept in complement M199 (1-3). Penile tissue was harvested, transferred to a sterile vial containing HBSS (Hank's balanced salt solution; Cat #14025092, Gibco) and washed twice with phosphate-buffered saline (PBS). The glans, urethra, and dorsal neurovascular bundles were removed from the penis and only cavernosal tissue was used for MCEC culture. Cells with a passage between 2 and 4 were used in all experiments.

Diabetes-induced angiopathy was mimicked with serum-starved cells overnight and exposed to high glucose (30 mM glucose) conditions for 72 hours at 37° C. in a humidified 5% CO₂ atmosphere (Diabetes 54, 2179-2187 (2005)). Normal glucose (5 mM glucose, Cat #G7021, Sigma-Aldrich) condition was used as a control.

### Example 1-2: Animals Used in Examples

Eight-week-old male C57BL/6 (Orient Bio, Korea) and LRG1-Tg male (Macrogen Inc., Seoul, Korea) mice were used. Mice of the same age were used in all experiments and a wild-type littermate was used as a control. The experiments were conducted under the approval of the Intuitional Animal Care and Use Committee (verification number: INHA 180523-570) at Inha University. Diabetes was induced by intraperitoneal injection of streptozotocin (STZ; 50 mg/kg body weight) for 5 consecutive days as previously described (The Journal of Sexual Medicine 6, 3289-3304 (2009)). Eight weeks after diabetes induction, mice were anesthetized by intramuscular injection of ketamine (100 mg/kg) and xylazine (5 mg/kg).

### Example 1-3: Expression and Purification of Recombinant Protein

For expression of the recombinant LRG1 protein, the BamHI and NotI sites of a modified pcDNA3.1 vector encoding a thrombin recognition sequence used for affinity purification were cloned with the human LRG1 (residues V36-Q347) genes, and then the Fc domain of human IgG. Recombinant LRG1 protein was transiently expressed in Freestyle 293-F cells (Invitrogen). The cells were transfected at a ratio of DNA:PEI (polyethylenimine; linear, MW 25000; Polysciences) of 1:4 and incubated at 37° C. in a humidified 8% CO₂ incubator for 4 days. Then, the cells were pelleted by centrifugation and the supernatant containing the Fc-fused LRG1 protein was loaded onto protein A resin (Cat #1010025, Amicogen). LRG1-Fc was eluted using 0.2M glycine buffer (pH 2.7) and neutralized with 1M Tris-HCl buffer (pH 9.0), and the sample buffer was exchanged with PBS through dialysis. The LRG1-Fc binding protein A resin was treated with thrombin (0.5% [v/v] in 20 mM Tris-HCl pH 8.0, 200 mM NaCl) overnight at 4° C. and LRG1 protein was eluted from untagged human LRG1. The eluted LRG1 was purified by size exclusion chromatography on a Superdex 200 increase 10/300 GL column (GE Healthcare Life Sciences) using a buffer containing 20 mM Tris-HCl (pH 8.0) and 200 mM NaCl. Fractions containing LRG1 protein were pooled and concentrated to 7.4 mg/ml for crystallization.

For the expression of Fc-tagged recombinant LPHN2 ectodomain variants, residues F26-Q95 (Lec domain), residues V135-P394 (Olf domain), residues F26-P394 (Lec-Olf domain) or residues F26-R796 (Lec-Olf-GAIN/GPS domain) were cloned into the BamHI/NotI site of a modified pcDNA3.1 vector encoding a thrombin recognition sequence, followed by a protein A tag or the Fc domain of human IgG for use in affinity purification. YFP-His-tagged human LRG1 (LRG1-YFP) and Fc-tagged LPHN2 ectodomain variants (Lec, OlF, Lec-Olf or Lec-Olf-GAIN/GPS) were produced using the Expi293F expression system (Thermo Fisher) in accordance with the instructions of the manufacturer.

The cells were transfected with expression plasmids using ExpiFectamine (Cat #A14524, Thermo Fisher) and incubated in a humidified 8% CO₂ incubator at 37° C. for 2 days. After centrifugation of the cultures, the supernatant was loaded onto Ni-NTA resin (Cat #30230, Qiagen) for LRG1-YFP and protein A resin (Cat #1010025, Amicogen) for Fc-tagged LPHN2 ectodomain variants. LRG1-YFP was eluted with 250 mM imidazole in 20 mM Tris-HCl (pH 8.0) and 200 mM NaCl, and untagged LPHN2 ectodomain variants were lysed by dendritic thrombin (0.5% [v/v] in 20 mM Tris-HCl pH 8.0, 200 mM NaCl) and then eluted overnight at 4° C. The eluted LRG1-YFP and LPHN2 ectodomain proteins were further purified by size exclusion chromatography on a Superdex 200 increase 10/300 GL column (GE Healthcare Life Sciences.) using a buffer containing 20 mM Tris-HCl (pH 8.0) and 200 mM NaCl. The molecular weight of the protein was evaluated by SDS-PAGE and Coomassie Brilliant blue R-250 staining (BIO-RAD) using standard methods.

### Example 1-4: Tube Formation Assay

Tube formation assay was performed in accordance with a conventionally known method (The Journal of Sexual Medicine 9, 1760-1772 (2012)). Approximately 100 μl of growth factor-reduced Matrigel (Cat #354230, Becton Dickinson) was seeded into a 48-well tissue culture plate at 4° C. After gelation at 37° C. for at least 30 minutes, HUVECs or MCECs were seeded on the gel at 4×10⁴ cells/well in 300 μl of M199 medium. The cells were immediately treated with 1 μg/ml of LRG1-Fc, LRG1, deglycosylated LRG1 (DG-LRG1), LRG1 variants (N269D, N325D, N269D/N325D), TGF-β1 (5 ng/ml), LRG1-Fc+TGF-β1 (5 ng/ml), LRG1-Fc+anti-TGF-β1 antibody (10 μg/ml) and/or LRG1-Fc+TGF-β1 (5 ng/ml)+anti-TGF-β1 antibody (10 μg/ml). The cells were monitored by phase-contrast microscopy for tube formation for 12 to 16 hours and the number of master junctions was counted and quantified in four separate blind experiments using Image J software. (National Institutes of Health [NIH] 1.34, http://rsbweb.nih.gov/ij/).

### Example 1-5: Cell Migration Assay

For transwell migration assays, 1×10⁵ HUVECs in 200 μl of serum-free M199 were seeded into the upper inserts (8-μm pores) of 12-well plates (Cat #353182, Corning), and 600 μl of M199 supplemented with 20% FBS was added to the lower chamber. HUVECs were treated with 1 μg/ml of Pc (control), 1 μg/ml of LRG1-Fc, 5 ng/ml of TGF-β1 (Cat #240-B, R & D Systems), 10 μg/ml of anti-TGF-β1 antibody (Cat #MAB1835, R & D Systems), 5 ng/ml of TGF-β1+10 μg/ml of anti-TGF-β1 antibody (pre-mixed overnight for neutralization), and 1 μg/ml of DG-LRG1. After culture at 37° C. for 28 hours, non-migrated cells on the upper surface of the insert were removed with a cotton swab. Migrated cells were fixed, stained with crystal violet (Cat #V5265, Sigma) and then eluted with 10% acetic acid. The number of migrated or invaded cells was counted in four random fields. Signals were visualized and digital images were obtained with a confocal fluorescence microscope (K1-Fluo; Nanoscope Systems, Inc.).

### Example 1-6: TriCEPS-Based Ligand Receptor Capture (LRC-TriCEPS)

Receptor candidates for LRG1 were identified using TriCEPS-based ligand receptor capture (LRC-TriCEPS; cat #P05203, Dualsystems Biotech) in accordance with the instructions of the manufacturer. 300 μg of recombinant LRG1 protein or transferrin (control ligand) was dissolved in 150 μl of 25 mM HEPES buffer (pH 8.2), and 1.5 μl of TriCEPS reagent was added to each sample, followed by incubation at 22° C. for 90 minutes with constant agitation. HEK293T cells (1.2×10⁸) were mildly oxidized by incubation in PBS (pH 6.5) along with 1.5 mM NaIO₄ for 15 minutes at 4° C. in the dark together with gentle rotation. The cells were washed twice for 5 minutes at 300×g and resuspended in two separate tubes containing 18 ml PBS with 1% FBS (pH 6.5). Then, 150 μl of TriCEPS-coupled transferrin or TriCEPS-coupled LRG1 was added to each tube, followed by incubation for 90 minutes at 4° C. with constant gentle agitation. After the coupling reaction was complete, the cells were collected and the cell pellet was transferred to Dualsystems for LC-MS/MS analysis.

### Example 1-7: shRNA Delivery Using Lentivirus (Knockdown)

After the animals were anesthetized with intramuscular injections of ketamine (100 mg/kg) and xylazine (5 mg/kg), the scrambled control shRNA (shCon) lentivirus particles (Cat #SC-108080, Santa Cruz) or SMART vector mouse lentivirus containing shRNA targeting LPHN2 (shLPHN2; Cat #V3SM7603-234963095, Dharmacon) were administered by intramuscular injection at a dose of 1 transforming units (TU)/mouse. For in vitro (HUVECs, HEK293T cells and MCECs) and ex vivo (MPGs and DEGs) cell culture experiments, shCon or shLPHN2 lentivirus particles were added to the culture medium at a concentration of 5×10⁴ TU/ml. The shRNA sequence targeting murine LPHN2 and the shRNA sequence targeting human LPHN2 are shown in Table 1 below. Experiments were performed 3 days after lentiviral infection.

TABLE 1

| | | SEQ ID |
|---|---|---|
| Item | Sequence (5′->3′) | NO: |
| Human LPHN2- | ACGGCTTATGGGTCATTTA | 3 |
| targetting shRNA | | |
| Mouse LPHN2- | TCAACTGCTAGGTCGATAT | 4 |
| targetting shRNA | | |

### Example 1-8: Cell Surface Binding Assay

HEK293T cells and HUVECs were plated on 8-well Lab-Tek chamber slides in standard growth medium. The cells were incubated with LRG1-YFP or YFP at 37° C. in a humidified 5% CO₂ environment for 1 hour and then washed twice with PBS and fixed with 4% formaldehyde in PBS for 15 minutes at room temperature. The wells were washed twice with PBS, and then the slides were mounted with ProLonged Antifade mounting solution (Cat #P10144, Molecular Probes). Fluorescently labeled LRG1 was observed under a confocal laser-scanning microscope (Zeiss LSM 780).

### Example 1-9: Immunoblots and Immunoprecipitation

Cells and tissues were lysed in RIPA buffer (Sigma-Aldrich) supplemented with protease inhibitors (Cat #P3100-001, GenDEPOT) and phosphatase inhibitors (Cat #P3200-001, GenDEPOT). Equal amounts of protein (30 μg per lane) from each whole-cell or tissue lysate were resolved by SDS-PAGE on 8 to 12% gels and transferred to polyvinylidene fluoride (PVDF) membranes. The membranes were blocked with 5% non-fat dried milk for 1 h at room temperature and then were incubated at 4° C. overnight with the following primary antibodies:

Anti-LPHN2 (Cat #ab209548, Abcam; 1:500), anti-LRG1 (Cat #HPA001888, Sigma; 1:500), anti-TGFβ1 (Cat #sc-146, Santa Cruz Biotechnology; 1:500), anti-phospho-eNOS (Cat #9571, Cell Signaling; 1:500), anti-eNOS (Cat #610297, Becton Dickinson; 1:1000), anti-phospho-Smad1/5 (Cat #9516, Cell Signaling; 1:500), anti-phospho-Smad2/3 (Cat #8828, Cell Signaling; 1:500), anti-BDNF (Cat #sc-546, Santa Cruz; 1:500), anti-NGF (Cat #sc-548, Santa Cruz; 1:500), anti-NT-3 (Cat #sc-547, Santa Cruz; 1:500), anti-phospho-Akt (Cat #9271, Cell Signaling; 1:500), anti-Akt (Cat #9272, Cell Signaling; 1:500), anti-phospho-PI3 kinase p85 (Cat #4282, Cell Signaling; 1:500), anti-PI3 kinase p85 (Cat #4292, Cell Signaling; 1:500), anti-phosphotyrosine clone 4G10 (Cat #05-321, Sigma; 1:500), anti-VEGF (Cat #sc-152, Santa Cruz; 1:100), and anti-ANG1 (Cat #sc-74528, Santa Cruz; 1:100). For immunoprecipitation, 1,000 μg of lysate was incubated with the indicated antibody (1-2 μg) for 3 to 4 hours at 4° C., followed by overnight incubation with Protein A/G PLUS-Agarose (Cat #SC-2003, Santa Cruz Biotechnology). Immunoprecipitates were washed five times with RIPA buffer, resolved by SDS-PAGE and immunoblotted with the indicated antibodies. TGF-β1 levels in conditioned medium (CM) were measured by first harvesting and centrifuging the CM at 1500 rpm for 5 min to remove cell debris and then precipitating CM samples with a trichloroacetic acid (TCA) and acetone mixture (10% TCA and 10 mM dithiothreitol [DTT] in acetone) at −20° C. overnight (Brain Res 1265, 158-170 (2009)). The precipitated proteins were washed twice with 20 nM DTT in acetone and lysed in RIPA buffer (Sigma-Aldrich) supplemented with protease inhibitors (Cat #P3100-001, GenDEPOT) and phosphatase inhibitors (Cat #P3200-001, GenDEPOT). Equal amounts of protein (30 μg per lane) were resolved by SDS-PAGE and immunoblotted with anti-TGF-β1 (Cat #sc-146, Santa Cruz Biotechnology; 1:500). Densitometric analyses of Western blot bands were performed using ImageJ 1.34.

### Example 1-10:LC-MS/MS Analysis of Immunoprecipitants

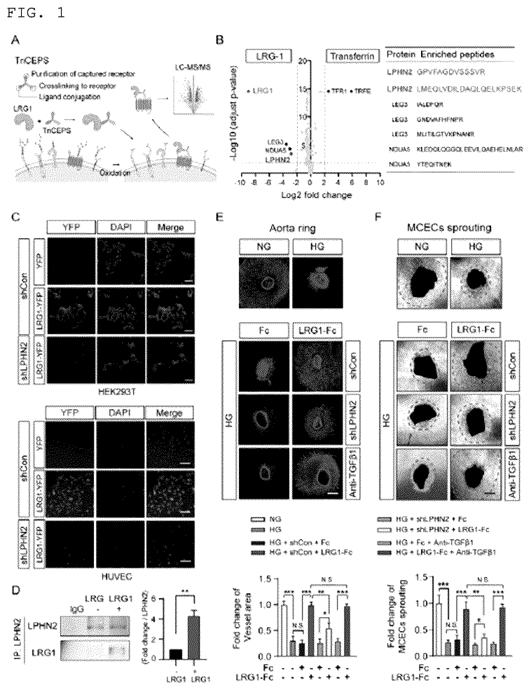

HUVECs were serum-starved for 6 hours and then treated with LRG1 (1 μg/ml) for 0, 10, 30, and 60 minutes. The HUVECs were lysed with RIPA buffer (Cat #89900, Sigma), and then total cell lysates were immunoprecipitated with LPHN2 antibody (Cat #sc-514197, Santa Cruz; 1:50) and analyzed by SDS-PAGE and Coomassie Blue staining. The indicated bands (Supplementary FIG. 1B, framed in red) were excised from the SDS-PAGE gels and Nano LC-MS/MS analyses were performed using an Easy n-LC (Thermo Fisher, San Jose, CA, USA) and LTQ Orbitrap XL mass spectrometer (Thermo Fisher) equipped with a nano-electrospray source (Yonsei Proteome Research Center, Korea). Samples were separated from a C18 nanobore column (150 mm×0.1 mm, 3 μm pore size, Agilent).

The mobile phase A for LC separation was 0.1% formic acid+3% acetonitrile in deionized water and mobile phase B therefor was 0.1% formic acid in acetonitrile. The chromatographic gradient was designed to achieve a linear increase from 0% B to 32% B in 23 minutes, from 32% B to 60% B in 3 minutes, 95% B in 3 minutes and 0% B in 6 minutes. The flow rate was maintained at 1,500 nl/min. Mass spectra were obtained by data-dependent collection using full mass scans (350-1800 m/z) and then 10 MS/MS scans. In MS1 full scans, the orbitrap resolution was 15,000 and the AGC was 2×10⁵. In MS/MS in LTQ, AGC was 1×10⁴. Peptide sequences present in the protein sequence database were identified using the Mascot algorithm (Matrix Science, USA). The database search criteria are as follows:

Taxonomy, Homo sapiens, Mus musculus; fixed modification, carbamidomethylated at cysteine residues; variable modification, oxidized at methionine residues; maximum allowed missed cleavages, 2; MS tolerance, 10 ppm; MS/MS tolerance, 0.8 Da.

Only trypsin-lysed peptides were considered and peptides were filtered with a significance threshold of P<0.05.

### Example 1-11: Aortic Ring Assay

Aortas harvested from 8-week-old C57BL/6 mice were placed on the 8-well Nunc Lab-Tek Chamber Slide System (Sigma-Aldrich) and kept in place with an overlay of 50 μl of Matrigel. Aortic rings were cultured in complete M199 for 5 days in normal glucose (5 mM) or high-glucose (30 mM) medium, with or without RG1-Fc (1 μg/ml), shCon or shLPHN2 (5×10⁴ TU/ml culture medium), and with or without anti-TGF-β1 antibody (10 μg/ml). Aortic segments and sprouting cells were fixed in 4% paraformaldehyde for at least 30 minutes and used for immunofluorescence analysis of PECAM-1 (Cat #MAB1398Z, Millipore; 1:50) (J Vis Exp, (2009)).

### Example 1-12: Ex Vivo Endothelial Cell Sprouting Assay

Mouse corpus cavernosum tissue was cut into two or three pieces and then plated on Matrigel stock solution-coated 6-well cell culture dishes. The Matrigel was incubated for 15 minutes at 37° C., and polymerized in 3 ml of normal-glucose (5 mM) or high-glucose (30 mM) conditioned complete M199, in the presence of 1 μg/ml of LRG1 and shRNA (with or without 1 μg/ml of shCon or LRG1). shLPHN2 (5×10⁴ TU/ml culture medium) and anti-TGF-β1 antibody (10 μg/ml) were added to the cell culture dish. Then, the cells were incubated at 37° C. in a humidified 5% CO₂ environment while the conditioned complete M199 was changed every 2 days. After 7 days, images were obtained with a phase-contrast microscope and the sprouting cell density was analyzed using ImageJ 1.34.

### Example 1-13: Measurement of Erectile Function

Erectile function was measured in a well-known method (The Journal of Sexual Medicine 6, 3289-3304 (2009)). A bipolar platinum wire electrode was placed around the cavernous nerve. Stimulation parameters were 1 or 5 V, the frequency was 12 Hz, the pulse width was 1 ms and the duration was 1 minute. Maximum intracavernous pressure (ICP) was recorded during stimulation. The total ICP was determined as the area under the curve from the start of the cavernous nerve stimulation to 20 seconds after the end of the stimulation. Each electrical stimulation was repeated at intervals of at least 10 minutes. Systemic blood pressure was measured using a non-invasive tail-cuff system (Visitech Systems) verified in previous studies (Hypertension 25, 1111-1115 (1995)). Systemic blood pressure was measured before measurement of ICP because vibrations during electrical stimulation may affect evaluation of blood pressure. The ratios of maximum ICP and total ICP to mean systolic blood pressure (MSBP) were calculated to reflect changes in systemic blood pressure. Physiological and metabolic parameters of control and STZ-induced diabetic mice are shown in Table 2.

TABLE 2

| | 2 weeks after treatment of indicated protein together | | | | |
|---|---|---|---|---|---|
| | with [symbol] hC on [symbol] virus (n = 6). | | | | |
| | | | STZ-induced diabetic mice | | |
| | Fc | LRG?-Fc | Fc | LRG [symbol] -Fc | LRG [symbol] -Fc [symbol] A [symbol] TGF [symbol] |
| Body weight (g) | [symbol] 0 [symbol] | 3 [symbol] | [symbol] 5 | 2 [symbol] | 24 [symbol] |
| Fasting glucose (mg/dl) | 10 [symbol] 2. [symbol] | 10 [symbol] 1.7 | [symbol] 2. [symbol] | [symbol] | 41 [symbol] |
| Post [symbol] and [symbol] glucose (mg/dl) | 172 [symbol] | 1 [symbol] | [symbol] | [symbol] 4 [symbol] | 55 [symbol] |
| M [symbol] BP (mm Hg) | 102 [symbol] 1 [symbol] | 103 [symbol] 2.2 | 102 [symbol] | 10 [symbol] 1. [symbol] | 107. [symbol] 3.3 |

2 weeks after treatment of indicated protein together
with [symbol] hLPHN2 [symbol] ivirus (n = 6).

STZ-induced diabetic mice

| | Fc | LRG?-Fc | Fc | LRG [symbol] -Fc | LRG [symbol] -Fc [symbol] A [symbol] TGF [symbol] |
|---|---|---|---|---|---|
| Body weight (g) | 4.7 [symbol] | [symbol] 5 | [symbol] 7 [symbol] 0.4 [symbol] | 2 [symbol] | 22.2 [symbol] |
| Fasting glucose (mg/dl) | 103. [symbol] | 10 [symbol] 2.2 | [symbol] 7 [symbol] | [symbol] .2 [symbol] | [symbol] 00 [symbol] |
| Post [symbol] and [symbol] glucose (mg/dl) | 1 [symbol] .1 | 1 [symbol] 14. [symbol] | [symbol] | [symbol] | [symbol] |
| M [symbol] BP (mm Hg) | [symbol] | 1 [symbol] | 102 [symbol] | 10 [symbol] | 1 [symbol] |

| | 8 weeks after induction of diabetes with STZ in LRG1- [symbol] mice (n = [symbol] ). | | | |
|---|---|---|---|---|
| | | | STZ-induced diabetic mice | |
| | | | LRG1- | |
| | WT | LRG1- [symbol] | Tg [symbol] | LRG1-Tg [symbol] 2 |
| Body weight (g) | [symbol] .4 [symbol] | 33.1 [symbol] | 2 [symbol] | 22. [symbol] |
| Fasting glucose (mg/dl) | 1 [symbol] 1 | 11 [symbol] | [symbol] | [symbol] |
| Post [symbol] and [symbol] glucose (m [symbol] ) | 153 [symbol] | 1 [symbol] 11. [symbol] | [symbol] 7 [symbol] | [symbol] |
| MSBP (mm [symbol] g) | 105 [symbol] 1. [symbol] | 11 [symbol] 1. [symbol] | 111 [symbol] | 11 [symbol] 2. [symbol] |

Values are [symbol] means ± SEM for n = [symbol] or [symbol] animals per group. [symbol] P [symbol] < [symbol] group. [symbol] epto [symbol] tocin [symbol] MSB [symbol] mean systolic blood pressure [symbol] [symbol]
indicates data missing or illegible when filed

### Example 1-14: MRG and DRG Explant Culture and Sprouting Assay

Mouse main pelvic ganglion (MPG) and dorsal root ganglion (DRG) tissues were dissected and maintained in accordance with conventional reported methods (BJU international 92, 631-635 (2003), Andrology 5, 327-335 (2017)). MPG tissue and L3-L5-derived DRG tissue adhered to the ventral part of the prostate were isolated from male mice using a microscope, transferred to a sterile vial containing HBSS, rinsed twice with PBS, and washed. MPG and DRG tissues were cut into small fragments and placed in an 8-well Nunc Lab-Tek chamber slide system coated with poly-D-lysine hydrobromide (Sigma-Aldrich). The MPG and DRG tissues were completely covered with Matrigel and the culture plate was incubated in a 5% CO₂ environment at 37° C. for 10 to 15 minutes. Then, 1 ml complete Neurobasal medium (Gibco) supplemented with 2% serum free B-27 (Cat #17504-044, Gibco) and 0.5 nM GlutaMAX-I (Cat #35-050-061, Gibco) was added to the plate and incubated in a humidified 5% CO₂ environment at 37° C. in normal glucose (5 mM) or high glucose (30 mM) medium with or without LRG1-Fc (1 μg/ml) and shRNAs (shCon or shLPHN2 (5×10⁴ TU/ml culture medium)) with or without an anti-TGF-β1 antibody (10 μg/ml). After culturing for 7 days, neurite outgrowth segments were fixed in 4% paraformaldehyde for 30 minutes or more and immunostained with an anti-BIII tubulin antibody (Cat #ab107216, Abcam; 1:100).

### Example 1-15: Primary Cortical Neuron Culture

Primary mouse cortical neurons were derived from E15 wild-type embryos obtained from timed-pregnant C57BL/6N mice (Orient Bio, Korea). Cortical tissues were dissected from embryos of both genders and washed three times with ice-cold Hanks' balanced salt solution (HBSS). Cortical pieces were lysed with 0.125% trypsin/EDTA (Cat #25200056, Gibco) at 37° C. for 5 minutes with periodic shaking. The trypsin/EDTA was neutralized by adding 5% FBS (Cat #10270106, Gibco) and cortical neurons were dissociated by gently pipetting 10 times. The resulting cell suspension was filtered through a 70 μm mesh (Cat #352350, Becton Dickinson) cell strainer and centrifuged at 800×g for 3 minutes. The result was resuspended in complete neurobasal medium (Cat #21103049, Gibco) supplemented with 2% serum-free B-27 (Cat #17504-044, Gibco) and 0.5 nM GlutaMAX-I (Cat #35-050-061, Gibco), and cortical neurons were cultured on coverslips pre-coated with poly-D-lysine hydrobromide (0.1 mg/ml; Cat #P0899, Sigma-Aldrich) and laminin (10 ng/ml; Cat #23017015, Gibco).

For immunofluorescence analysis, the cells were plated at a density of 1×10⁵ cells/well on 24-well plates and treated for 5 days in accordance with the following process:
After primary spreading, the cells were infected with lentivirus on the 1st day
After primary spreading, the cells were treated twice with an antibody, protein or inhibitor on the 2nd and 4th days
Sprouted axons were fixed in 4% paraformaldehyde for 30 minutes or longer and then incubated with 0.3% Triton X-100 for 5 minutes to allow cells and axons to permeate.
Immunostaining was performed using BIII tubulin antibody (Cat #ab107216, Abcam; 1:100)

For quantification, the cells were quantified at 200 cells/field using Image J. For Western blot analysis, the cells were seeded at 2×10⁶ cells/well in 6-well plates and treated with lentivirus or antibodies, proteins and/or inhibitors.

### Example 1-16: BrdU Labeling

BrdU (50 mg/kg body weight; Cat #19-160, Sigma-Aldrich) was intraperitoneally injected once daily for 3 consecutive days into mice of each group (control and STZ-induced diabetic mice), 2 weeks after repeated intracavernosal injection with Fc or LRG1-Fc (5 μg/20 μl) on day 0 and day 3. After one day, the mice were sacrificed. An additional antigen search step was performed using the 5′-bromo-2′-deoxyuridine antibody (BrdU; Cat. MCA2060, AbD Serotec; 1:50). The number of BrdU-positive endothelial cells was counted in four different fields and the cells were expressed as the number of cells/high-power field.

### Example 1-17: TUNEL Assay

Apoptosis in cavernous tissue from control and STZ-induced diabetic mice was evaluated using TUNEL (terminal deoxynucleotidyl transferase-mediated deoxyuridine triphosphate nick-end labeling) assays. The ApopTag Fluorescein In Situ Apoptosis Detection Kit (Cat #S7160, Chemicon) was used in accordance with the instructions of the manufacturer. Digital images and the numbers of apoptotic cells were determined using a confocal fluorescence microscope. For in vitro studies, the number and percentage of TUNEL-positive cells were evaluated.

### Example 1-18: Histological Examination of Immunofluorescence-Stained Tissue

Penile tissue was fixed in 4% paraformaldehyde for 24 hours at 4° C., then frozen and cut into sections (12 μm).

Cultured MPG tissues, DRG tissues, and aortic rings were fixed in 4% paraformaldehyde at room temperature for 10 minutes and then incubated with blocking solution at room temperature for 2 hours. Then, the samples were incubated overnight at 4° C. with the following primary antibodies:
Anti-LPHN2 (Cat #ab138498, Abcam; 1:100),
Anti-PECAM-1 (Cat #MAB1398Z, Millipore; 1:50),
Anti-NG2 (Cat #AB5320, Millipore; 1:50), and
Anti-βIII tubulin (Cat #ab107216, Abcam; 1:100).

The samples were washed several times with PBS and were incubated with tetramethylrhodamine (TRITC)-conjugated donkey anti-chicken secondary antibodies (Cat #703-025-155, Jackson ImmunoResearch Laboratories; 1:200), fluorescein isothiocyanate (FITC)-conjugated goat anti-Armenian hamster secondary antibodies (Cat #127-095-160, Jackson ImmunoResearch Laboratories; 1:200), or DyLight 550-conjugated donkey anti-rabbit secondary antibodies (Cat #ab98489, Abcam; 1:200) at room temperature for 2 hours. The signals were visualized and digital images were obtained using a confocal fluorescence microscope (K1-Fluo; Nanoscope Systems, Inc.). Immunofluorescence staining intensity was quantified using ImageJ 1.34.

### Example 1-19: Solid-Phase Binding Assay

Solid phase binding analysis was performed by a known method (Mol Cancer Ther 14, 470-479 (2015)). LPHN2 extracellular domain variants (Lec, Olf, GAIN/GPS domain; 100 nM) were added to MaxiSorp 96-well plates (Nunc) and incubated at room temperature for 1 hour. The wells were washed twice with PBS and then incubated with 1% bovine serum albumen (BSA) for 2 hours. After blocking, various amounts (0.001, 0.005, 0.01, 0.05, 0.1, 0.5, 1, 5 or 10 μM) of wild-type LRG1 or DG-LRG1 were added to 96-well plates coated with the indicated LPHN2 extracellular domain variants. LRG1 bound to the coated protein was detected by ELISA using an anti-LRG1 antibody (Cat #sc517443, Santa Cruz) and a peroxidase-conjugated anti-mouse secondary antibody (Cat #62-6520, Thermo Fisher Scientific).

### Example 1-20: Binding Affinity of LRG1 to HUVEC and HEK293T Cells

The binding affinity of LRG1 to normal (parental) and LPHN2 knockdown HUVEC and HEK293T cells was measured at 1.0×10⁶ cells/200 μl using 1 μM of Alexa 647-conjugated LRG1 or Alex647-conjugated DG-LRG1. The cells were washed with PBS and then LRG1 or DG-LRG1 bound to the cell surface was detected by monitoring fluorescence signals using fluorescence-activated cell sorting (FACS) and analyzed using FlowJo 10 software (FlowJo, LLC).

### Examples 1-21: Crystallization and Structural Analysis

Crystals were grown at 291K by a hanging-drop vapor diffusion method using in a mixture of 1 μl of LRG1 protein (7.4 mg/ml) with 1 μl of crystallization buffer (200 mM lithium sulfate, 100 mM sodium acetate pH 4.5, 48% PEG400 [v/v]) (BIODESIGN 8, 60-63 (2020)). For data collection at 100K, the crystals were immersed in cryoprotection buffer (200 mM lithium sulfate, 100 mM sodium acetate pH 4.5, 48% PEG400 [v/v], 30% glycerol [v/v]) and then flash-frozen in liquid nitrogen.

Diffraction data were collected at the 7A beam line of the Pohang Accelerator Laboratory and processed using the HKL2000 program (Methods Enzymol 276, 307-326 (1997)). The LRG1 crystal belongs to the space group P6322 and has the following unit cell dimensions:

a=143.0 Å; b=143.0 Å; c=113.7 Å; α=90°; β=90°; and γ=120°.

The initial phases were calculated by molecular replacement using PHASER22 (J Appl Crystallogr 40, 658-674 (2007)) and the structure of NGL3 (Protein Data Bank code: 3ZYN) was used as a search probe for structure determination. The atomic model was built after iterative rounds of model building using the program COOT (Acta Crystallogr D 60, 2126-2132 (2004)) and refinement using the program PHENIX24 Acta Crystallogr D 58, 1948-1954 (2002)). The final model was validated using MolProbity in PHENIX (Rwork=18.58%/Rfree=21.94%. A Ramachandran plot analysis of the LGR1 structure showed that 92.51% and 0.00% of residues were in favored and outlier regions, respectively. The structure factor and coordinate files have been deposited in the Protein Data Bank under accession code: 7D67).

### Example 1-22: Cignal Finder GPCR Signaling 10-Pathway Reporter Array and Phospho Array

For Cignal Finder GPCR signaling 10-pathway reporter array analyses, HUVECs (5×10³ cells/well) diluted in 80 μl of Opti-MEM supplemented with 5% FBS and 0.1 mM nonessential amino acids were transfected with the pre-incubated transfection mixture and then incubated at 37° C. in a 5% CO₂ environment for 24 hours. The pre-incubated transfection mixture was obtained by incubation in 0.5 μl of Lipofectamine 2000 (Invitrogen) and reporter DNA constructs (Cignal Finder Reporter Array for GPCR signaling 10-Pathway; CCA-109L-2, Qiagen) at room temperature for 30 minutes. After serum starvation for 4 hours, reporter DNA-transfected HUVECs were treated with 1 μg/ml of LRG1 or DG-LRG1 for 6 hours. Then, 10 intracellular signaling pathway reporters were analyzed by quantifying the luminescent signal using the Cignal 10-Pathway Reporter Array kit in accordance with the instructions of the manufacturer. Expression values were normalized by setting a sample ratio that distinguished between genuine cellular responses (firefly luminescence signal) and non-specific responses (Renilla luminescence signal), and plotted as fold-change of relative luminescence signals.

For the phospho array, HUVECs were serum-starved for 8 hours and then incubated with anti-TGF-β1 antibody for 1 hour, after which HUVECs were stimulated with 1 μg/ml of LRG1 for 30 minutes. Whole-cell lysates of HUVECs were prepared, and aliquots of lysates containing 50 μg of protein were used for phospho explorer antibody assays using an antibody array assay kit (Cat #KAS02, Fullmoon Biosystems, Sunnyvale, CA, USA) in accordance with the manufacturer's instructions. The phospho explorer antibody array (Full Moon Biosystems, Inc.) consisted of 1,318 antibodies and each antibody was present as two replicates printed on a coated glass microscope slide, as well as multiple positive and negative controls. Phospho explore antibody array experiments and analyses were performed as a custom service by E-Biogen (Ebiogen Inc., Seoul, Korea).

### Example 1-23: Network Analysis

First, proteins with an increased phosphorylation level of 25% or more by DG-LRG1 treatment were identified. Then, 10 protein-protein interactions (PPI) of phosphorylated proteins were obtained from 10 interactome databases including BioGRID (Nucleic Acids Res 34, D535-539 (2006)), HuRI (Nature 580, 402-408 (2020)), IntAct (Nucleic Acids Res 32, D452-455 (2004)), HitPredict (Nucleic Acids Res 39, D744-749 (2011)), IID (Nucleic Acids Res 47, D581-D589 (2019)), MINT (Nucleic Acids Res 35, D572-574 (2007)), DIP (Nucleic Acids Res 30, 303-305 (2002)), HPRD (Nucleic Acids Res 37, D767-772 (2009)), HTRIdb (BMC Genomics 13, 405 (2012)), and STRING (Nucleic Acids Res 43, D447-452 (2015)). A network model describing the interactions between the identified phosphoproteins was established using the obtained PPIs. A number of proteins were added to the network model for better understanding of the activation of DG-LRG1-related signaling pathways. In the network model, the phosphorylated proteins were aligned based on localization and activation/inhibition information obtained from the KEGG pathway database (Nucleic Acids Res 28, 27-30 (2000)).

### Example 1-24: Statistical Analysis

Unless otherwise mentioned, data are expressed as mean±SEM obtained through at least three independent experiments. Student's t-test was used to determine significant differences between the two groups using Prism 8 software (Graph Pad Software). P-values<0.05 were considered statistically significant. Each P-value is marked with an asterisk in the figure (*P<0.05, **P<0.01, ***P<0.001).

### Example 2: Identification of LPHN2 as Novel Receptor Independent of TGF-β of LRG1

Conventionally, vascular and nerve regeneration effects based on TGF-β of LRG1 and the co-receptor endoglin (ENG) thereof have been known, but due to the weak binding affinity (˜2.9 μM) between LRG1 and ENG, it is difficult to fully explain the vascular and nerve regeneration effects only based on the TGF-β-based mechanism. Therefore, the present inventors predicted that there would be another angiogenic and neurogenic pathway of LRG1 in addition to the TGF-β/ENG-based pathway.

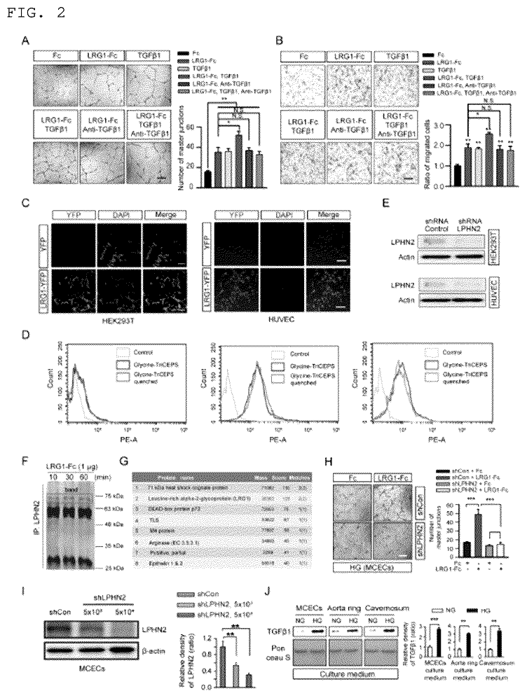

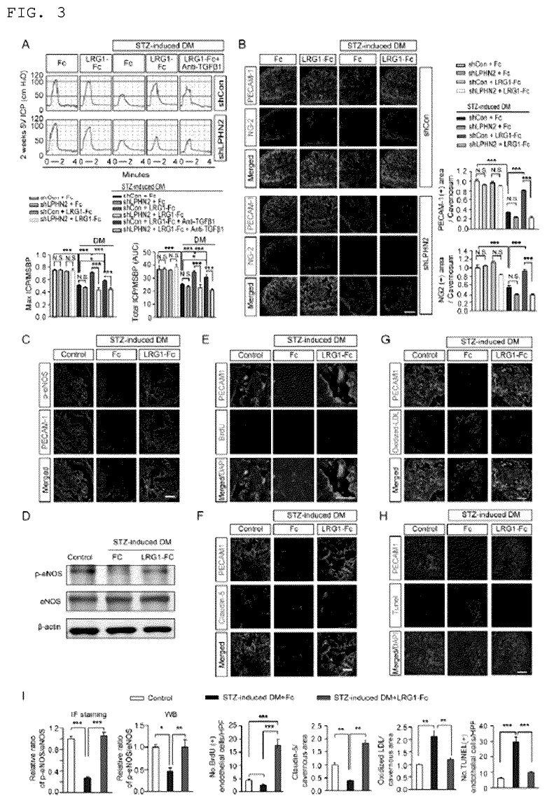

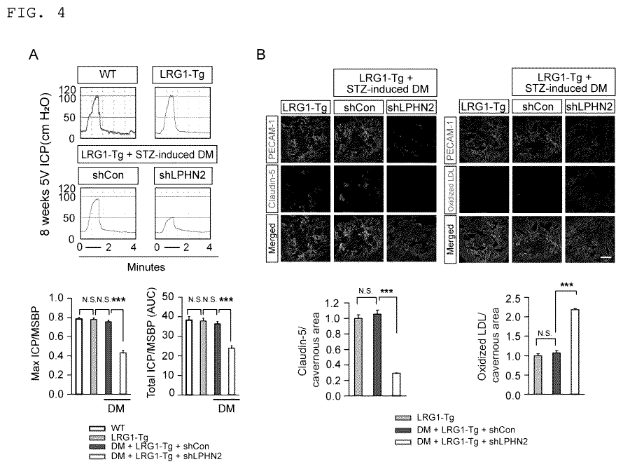

A proteomics approach was used to identify the TGF-β1-independent LRG1 receptor. First, LRG1 bound to the cell surface of the HUVECs and HEK293T cells was identified, which indicates that an unknown cell surface receptor of LRG1 is present in both cell lines (FIG. 2C). Ligand-based receptor capture (LRC) was performed in viable HEK293T cells, which can be cultured at large-scale, to identify novel LRG1 cell receptors. To this end, TriCEPS, a chemoproteomic reagent having three moieties for ligand conjugation, and receptor capture and purification, which enabled the identification of a glycosylated target receptor, was used (Nat Protoc 8, 1321-1336 (2013)).

Specifically, HEK293T cells oxidized with sodium metaperiodate were incubated with TriCEPS-coupled LRG1 and then the captured glycoprotein was affinity-purified and identified by LC-MS/MS (liquid chromatography-tandem mass spectrometry) analysis (FIGS. 1A and 2D).

The following LRG1 binding partner candidates were obtained based on the selection criteria (enrichment factor >4, false-discovery rate [FDR]-adjusted P-value<0.05) (FIG. 1B):
LEG3 (galectin-3),
NDUA5 (NADH dehydrogenase 1 alpha subcomplex subunit 5)
LPHN2 (latrophilin-2)

Among the three binding partner candidates, LPHN2 was selected as the most potent candidate for the LRG1 receptor because it is the only membrane protein expressed in both HUVEC and HEK293T cells (FIG. 2E). In particular, LPHN2 is an adhesion G-protein coupled receptor (GPCR), also known as calcium-independent alpha-latrotoxin receptor 2 (CIRL2) (J Biol Chem 273, 32715-32724 (1998)), which has recently been reported to mediate bidirectional signaling in synapse formation (Science 363, (2019)).

It was found that cell surface binding of LRG1-YFP was greatly reduced in both LPHN2 knockdown HUVECs and HEK293T cells compared to control cells treated with scrambled small hairpin RNA (shRNA) lentivirus (shControl) (FIGS. 1C and 2E). Moreover, LPHN2 immunoprecipitation and LC-MS/MS in HUVECs treated with soluble LRG1 and LC-MS/MS showed co-immunoprecipitation of LPHN2 and LRG1 (FIGS. 1D and 2F to 2G) and thus the specific and direct association of LRG1 and LRG1 in the HUVEC cell membrane.

When DM is not well controlled, hyperglycemia may irreversibly damage the cavernous nerve and blood vessels, leading to ED. Endothelial dysfunction in DM causes injured endothelial cell (EC) proliferation and migration (Int J Vasc Med 2012, 918267 (2012)). As expected, high concentrations of glucose inhibited in vitro tube formation of mouse cavernous endothelial cells (MCEC) and reduced microvessels growing in aortic rings and penile cavernosum tissues (FIGS. 1E to 1F and 2H).

However, all phenotypes induced by high glucose were completely restored by LRG1-Fc treatment and lentiviral-mediated LPHN2 knockdown significantly reduced the LRG1-dependent effects (FIGS. 1E and 1F, FIGS. 2H and 2I) Consistent with the previous report (Sci Rep 8, 11920 (2018)) the levels of secreted TGF-β1 proteins secreted from MCEC, aortic ring, and corpus cavernosum tissue were slightly increased under high-glucose culture medium conditions (FIG. 2J). However, LRG1-Fc effectively promoted angiogenesis even in the presence of a TGF-β1 blocking antibody (FIGS. 1E and 1F).

These results showed that LPHN2 is a TGF-β-independent cell surface receptor for LRG1 and direct binding between LRG1 and LPHN2 under high-glucose conditions is a novel mechanism pathway promoting angiogenesis.

### Example 3: Effects of Binding of LRG1 to LPHN2 on Induction of Angiogenesis and Alleviation of Erectile Dysfunction in STZ-Induced Diabetic Mice

The present inventors have reported that intracavernous delivery of recombinant COMP-Ang1, an angiogenic protein, can restore erectile function in a STZ-induced diabetic mouse model (hereinafter referred to as “diabetic mouse”) by enhancing endothelial regeneration (Diabetes 60, 969-980 (2011), Sci Rep 5, 9222 (2015)). Based on this, the erectile function of diabetic mice after intracavernous injection of LRG1-Fc was evaluated to determine the LRG1-dependent effect on angiogenesis in vivo. A ratio of maximal intra-cavernous pressure (ICP) or total intra-cavernous pressure (total ICP) to mean systolic blood pressure (MSBP) was greatly decreased by electrical stimulation of the cavernous nerve in diabetic mice compared to control mice.

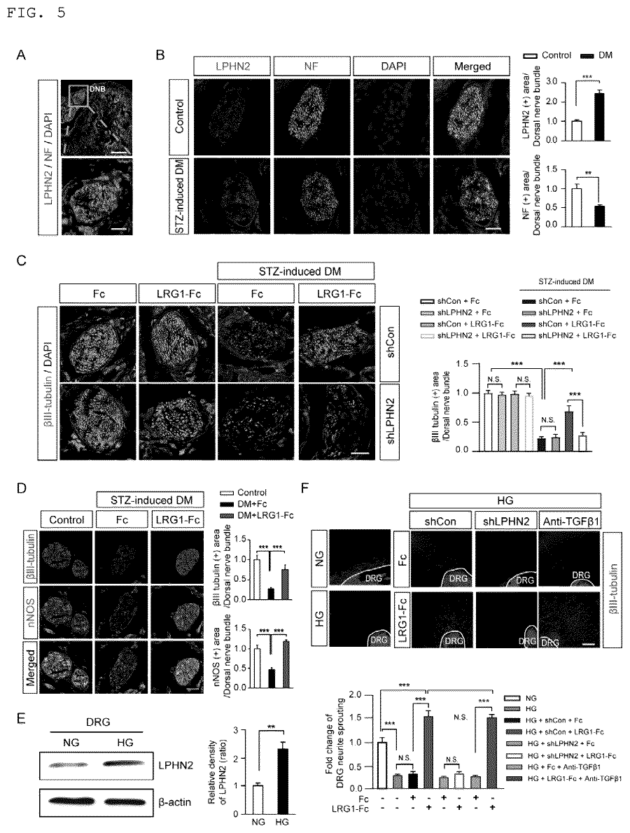

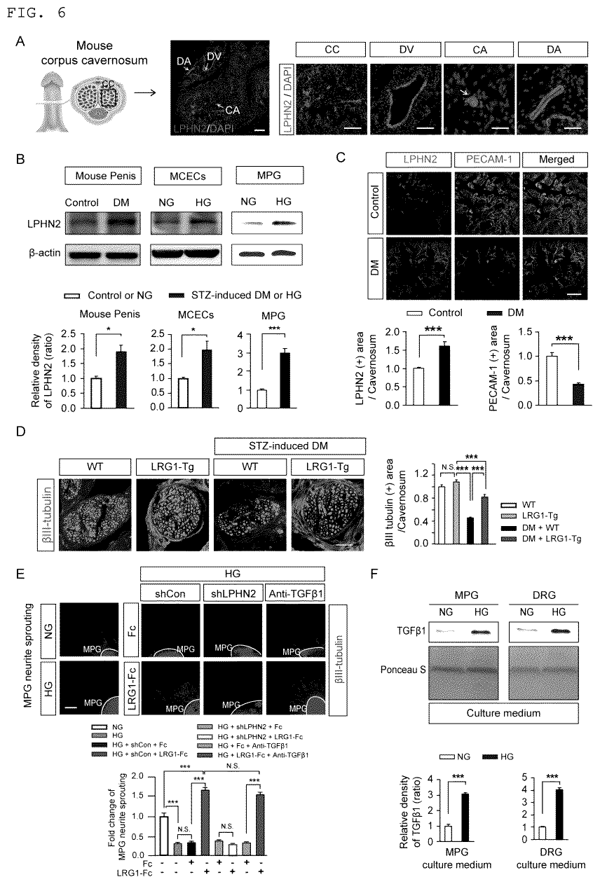

However, LRG1 injection restored erectile parameters to about 92% of the control value. The LRG1-dependent recovery effect was significantly eliminated by infection with shLPHN2 lentivirus, whereas infection with shControl lentivirus had no significant effect (FIG. 3A and Table 2).

In addition, the recovery of LRG1-mediated erectile function was slightly reduced in the presence of TGF-β1 blocking antibody, whereas additional infection with shLPHN2 lentivirus significantly reduced LRG1-mediated erectile function (FIG. 3A). Consistent with these results, erectile function was not impaired in LRG1-Tg mice even after multiple STZ injections, but was eliminated upon shRNA-mediated LPHN2 knockdown (FIG. 4A).

Similarly, immunofluorescence staining of corpus cavernosum tissue with PECAM-1 (platelet/endothelial cell adhesion molecule-1) and pericyte marker NG-2 (neuron-glial antigen 2) showed that the decrease in the contents of cavernous endothelial cells and diabetic periphery cells in STZ-induced diabetic mice was restored by LRG1, whereas it was not restored under the co-administration condition of shLPHN2 lentivirus and LRG1 (LRG1+shLPHN2 group) (FIG. 3B).

Expression levels of PECAM-1 and NG-2 were similar in wild-type mice infected with both shControl and shLPHN2 lentivirus, regardless of the presence of LRG1. In contrast, treatment of diabetic mice with LRG1 increased endothelial NO synthase phosphorylation (eNOS) (FIGS. 3C to 3D), increased endothelial cell proliferation (BrdU incorporation assay; FIGS. 3E and 31) and increased expression of claudin-5, a junction protein of endothelial cells (FIGS. 3F and 3I). On the other hand, cavernous endothelial permeability (extravasation of oxidized-LDL, FIGS. 3G and 31) and apoptosis (TUNEL assay, FIGS. 3H and 31) were decreased. Similar to the erectile function model (FIG. 4A), cavernous endothelial cell content and permeability were maintained in LRG1-Tg mice even after multiple STZ injections, but were eliminated under LPHN2-knockdown conditions (FIG. 4B).

### Example 4: Effects of Binding of LRG1 to LPHN2 on Induction of Neurite Outgrowth and Peripheral Nerve Regeneration in Hyperglycemia

Endothelial dysfunction and autonomic neuropathy are pathophysiological hallmarks of diabetic erectile dysfunction (Neurol Res 23, 651-654 (2001), Int J Impot Res, 129-138 (2007)). Considering the fact that the LPHN family plays an important role in growth cone migration and synapse formation (Front Neurosci 13, 700 (2019), Elife 9, (2020)), it was expected that the LRG1/LPHN2 axis might also affect cells and/or peripheral nerves of penile tissue.

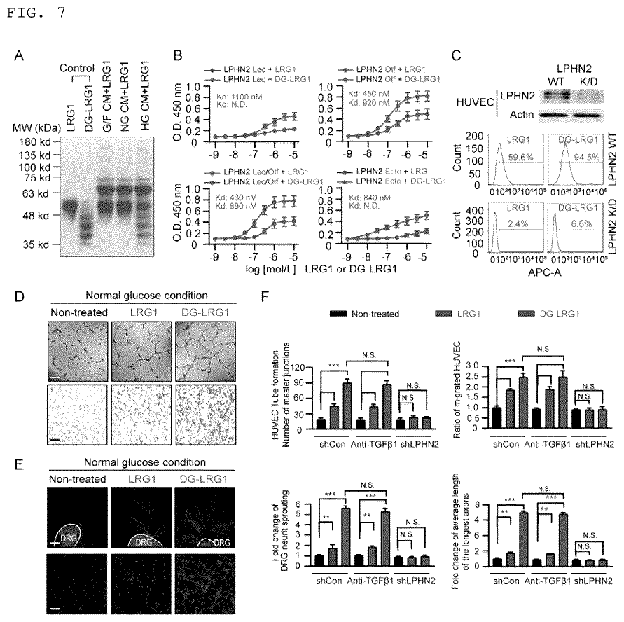

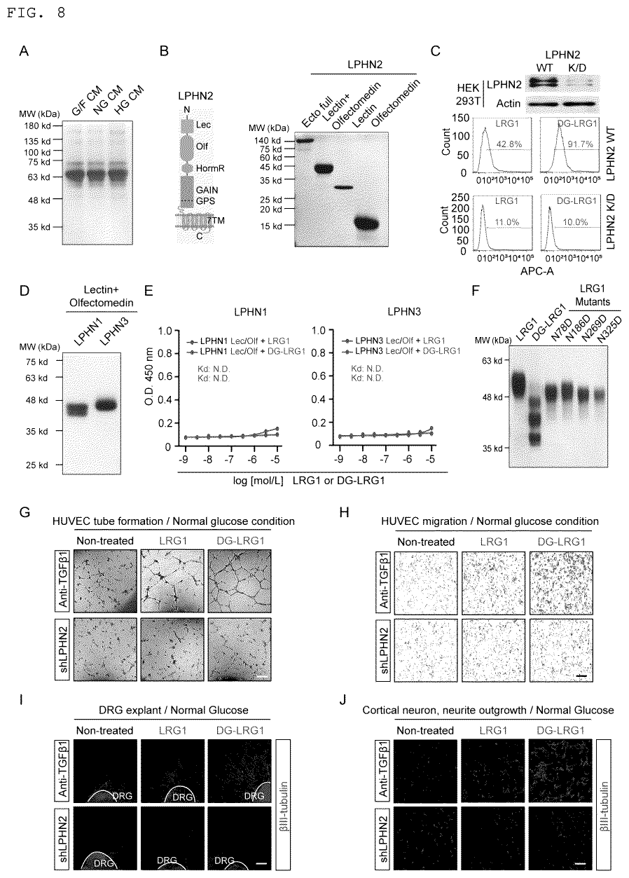

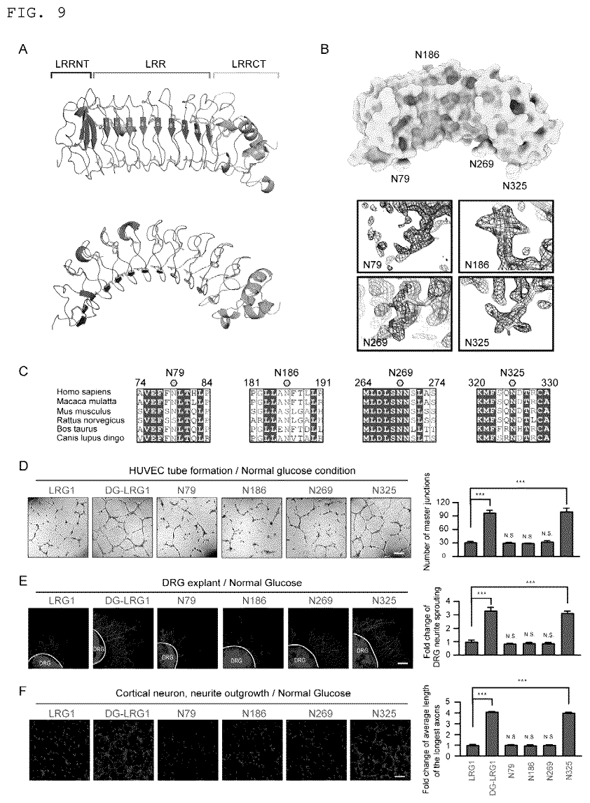

Immunofluorescent staining using an anti-LPHN2 antibody showed that LPHN2 is highly expressed in the dorsal vein (DV), the cavernous artery (CA), dorsal artery (DA), and the dorsal nerve bundle (DNB), similar to the vascular structure of the corpus cavernosum (CC) tissue (FIGS. 5A and 6A). Interestingly, LPHN2 expression in DNB, whole penile tissue and MCEC was significantly increased under diabetic and high glucose conditions (FIG. 5B and FIGS. 6B to 6C). On the other hand, neurofilament (NF) expression in DNB was significantly reduced in diabetic mice. Surprisingly, intracavernous injection of LRG1 completely restored penile neuronal contents and neuronal NOS (nNOS)-containing nerve fibers in diabetic mice, but these LRG1-dependent effects were eliminated by LPHN2 knockdown (FIGS. 5C and 5D). Similar to the cavernous endothelial cell content (FIG. 4B), the neuronal content of DNB was preserved in LRG1-Tg mice under STZ-induced diabetic conditions (FIG. 6D). These results suggest that LRG1 promotes penile nerve regeneration in diabetic mice.

Defective neurite outgrowth and impaired axonal regeneration are extensively associated with diabetic peripheral neuropathy (Front Endocrinol (Lausanne) 8, 12 (2017)). Therefore, whether or not LRG1 affects axonal generation in the main pelvic ganglion (MPG, a mix of parasympathetic and sympathetic ganglia that stimulate the pelvic organs) and dorsal root ganglion (DRG, a cluster of cells that transfer sensory information to the brain) was determined.

Specifically, MPG and DRG mouse explants were exposed to hyperglycemia and neural tissues were analyzed using BIII-tubulin immunofluorescence staining. Consistent with the previous report, high glucose conditions significantly reduced neurite outgrowth in both MPG and DRG explants (FIGS. 5F and 6E). On the other hand, treatment with LRG1-Fc stimulated neurite outgrowth in MPG and DRG hyperglycemic explants, which was eliminated by lentiviral-mediated LPHN2 knockdown (FIGS. 5F and 6E). Although high-glucose conditions increased TGF-β1 and LPHN2 expression in both MPG and DRG explants (FIGS. 5E, 6B and 6F), no LRG1-related neurotrophic effects were affected by TGF-β1 blocking antibodies. Overall, these data suggest that LRG1-induced neurite outgrowth is due to the TGF-β1-independent LPHN2 pathway.

### Example 5: Importance of LRG1 Deglycosylation Under High Glucose Condition for LRG1 and LPHN2 Binding

The data of the examples show that LRG1 promotes both angiogenesis and neurotization in diabetic mice, but has no significant effect on blood vessels or neurons in the penile tissue of control mice. Therefore, the presence of a functional activation mechanism in LRG1 under high glucose conditions was predicted. In order to verify this, LRG1 was cultured in no-glucose, normal glucose or high glucose (G/F, NG, HG) HUVEC culture medium, and samples were analyzed by SDS-PAGE.

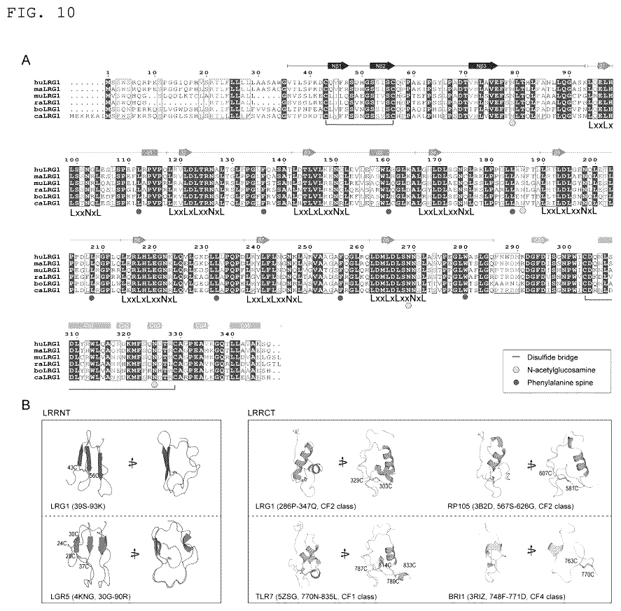

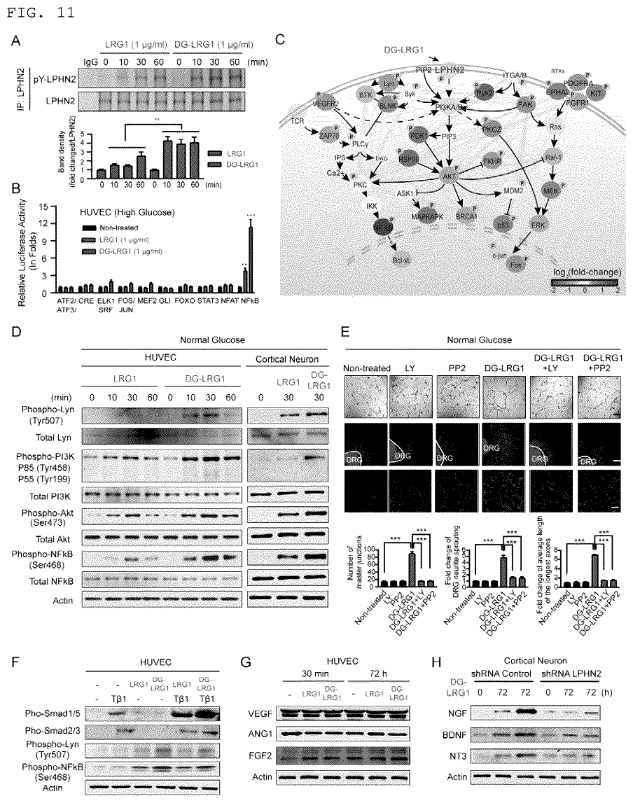

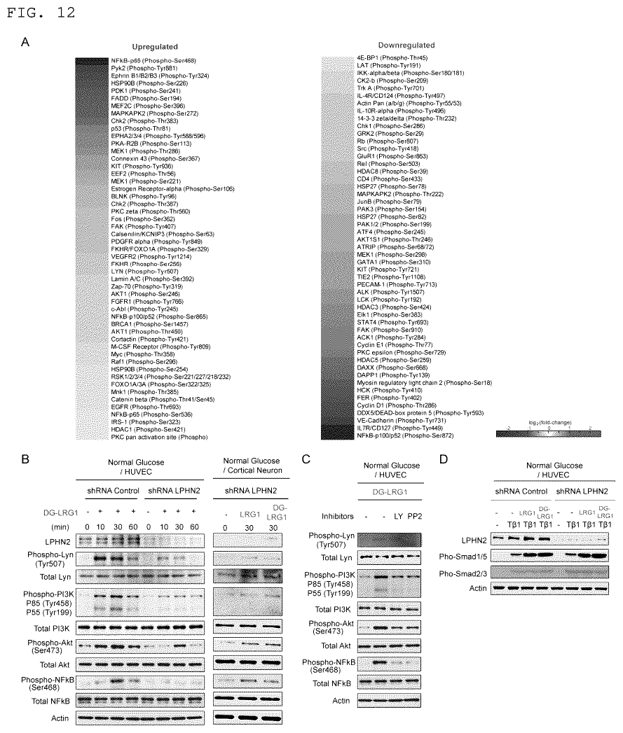

The result showed that the molecular weight shifted from 50 kDa to ˜30-40 kDa only when LRG1 was cultured in HG medium (FIGS. 7A and 8A). Considering that LRG1 is a glycoprotein, this change was predicted to be caused by deglycosylation of LRG1 under high glucose conditions. To verify this, LRG1 treated with PNGase F (N-glycan isolating enzyme) in parallel with LRG1 cultured in high-glucose conditioned medium was subjected to SDS-PAGE. In fact, the two bands were observed to be coincident, which indicates that deglycosylation of LRG1 was induced by HUVECs under high glucose culture conditions.

Whether or not deglycosylation of LRG1 (DG-LRG1) affects the binding affinity to LPHN2 in vitro (FIG. 7B) and ex vivo (FIGS. 7C and 8C) was determined. Solid-phase binding assays using LRG1 (wild-type and deglycosylated forms) and extracellular domains of LPHN2 showed that the Olf domain of LPHN2 was the minimal binding domain to LRG1, and the binding affinity of LRG1 to LPHN2 Lec/Olf domains (KD=920 nM) was found to be significantly increased (KD=450 nM) by deglycosylation of LRG1 (FIGS. 7B and 8B). Cell surface binding rates of LRG1 and DG-LRG1 in HUVEC and HEK293T cells were evaluated and verified again (Table 3, FIGS. 7C and 8C)

TABLE 3
Cell surface binding rates of LRG1 and DG-LRG1

| | HUVEC | HEK293T |
|---|---|---|
| LRG1 | 59.6% | 42.8% |
| DG-LRG1 | 94.5% | 91.7% |

However, LRG1 and DG-LRG1 cell surface binding was significantly reduced in LPHN2-knockdown HUVECs and HEK293T cells (FIGS. 7C and 8C). Unlike cell surface binding, wild-type LRG1 or DG-LRG1 binds weakly to recombinant ecto-full LPHN2 (FIG. 7B). This suggests that proper protein treatment of the LPHN2 GPS motif in the cell membrane may be important for LRG1 binding and LPHN2-mediated cellular function (Handb Exp Pharmacol 234, 83-109 (2016)). Interestingly, it was found that LRG1 and DG-LRG1 could not bind to the Lec/Olf domains of LPHN1 and LPHN3, which had 87% sequence similarity to the Lec/Olf domains of LPHN2 (FIGS. 8D and 8E).

### Example 6: Angiogenic and Neurotrophic Effects of Deglycosylated LRG1 (DG-LRG1)

The angiogenic and neurotrophic effects of DG-LRG1 on central neurons (mouse cortical neurons) and HUVEC and DRG explants were determined under normal glucose conditions. Unlike native LRG1, DG-LRG1 significantly facilitated tube formation and cell migration of HUVECs (FIGS. 7D and 7F, and FIGS. 8G and 8H), axonal sprouting from DRG explants, and neurite outgrowth (FIGS. 7E, 7F, 81 and 8J) in cultured cortical neurons, regardless of the presence or absence of TGF-β1 blocking antibody. However, LPHN2 knockdown using shLPHN2 lentivirus in HUVECs, DRG explants, and cultured cortical neurons significantly reduced the aforementioned facilitating effect (FIG. 7F and FIGS. 8G to 8J). The results indicate that deglycosylation of LRG1 in hyperglycemia is essential for high-affinity binding to LPHN2 and is a mechanism that improves angiogenic effects in endothelial cells and neurotrophic effects in both peripheral and central neurons.

### Example 7: Identification of Main Glycosylation Sites Involved in Crystal Structure and Functions of LRG1

Leucine-rich repeat (LRR) domains are generally known to have a horseshoe shape and to be important structural motifs in the formation of protein-protein interactions (Cell Mol Life Sci 65, 2307-2333 (2008), Proc Natl Acad Sci USA 108 Suppl 1, 4631-4638 (2011)). To verify the effects of glycans of LRG1 on function, human LRG1 (NCBI accession number: NP_443204, residues V36-Q347) was crystallized and the structure thereof was determined at a resolution of 2.5 Å (FIG. 9A).

Crystallographic information is shown in Table 4.

TABLE 4

| | human LRG1 |
|---|---|
| Data collection | |
| Space group | P6 [symbol] 22 |
| Cell dimensions | |
| a, b, c ( [symbol] ) | 143.02, 143.02, 113.73 |
| α, β, γ, (°) | 90, 90, 120 |
| Resolution ( [symbol] ) | 20-2.50 (2.59-2.50) [symbol] |
| R [symbol] or R_{merge} (%) [symbol] | 14. [symbol] (10.3) |
| [symbol] / [symbol] l | 46.4 (10.3) |
| Completeness (%) | 100.0 (100.0) |
| Redundancy | 3 [symbol] . [symbol] (35.3) |
| Refinement | |
| Resolution (Å) | 20.0-2. [symbol] 0 |
| No. reflections | 24066 |
| R [symbol] /R [symbol] (%) [symbol] | 18.58/21.94 |
| Average B-factor ( [symbol] ²) | 38.57 |
| R.M.S. deviations | |
| Bond length ( [symbol] ) | 0.01 |
| Bond angle (°) | 1.24 |
| Ramachandran favored (%) | 92.51% |
| Ramachandran outliers (%) | 0.00% |
| PDB entry | 7 [symbol] 7 |

[symbol]

Values in parentheses refer to the highest resolution [symbol] he [symbol] . [symbol]
R_{merge} = [symbol] Σ [symbol] Σ [symbol] Σ [symbol] Σ [symbol] [symbol]
R [symbol] Σ [symbol] Σ [symbol] [symbol]
indicates data missing or illegible when filed

LRG1 was determined to have a horseshoe-shaped solenoid structure containing eight central LRR repeats (LRR1-LRR8) between the N-terminus (LRRNT) and C-terminus (LRRCT) on both sides thereof (FIG. 9A). The result of sequence alignment of mammalian LRG1 identified key residues that were highly conserved (FIG. 10A):
common leucine residues and asparagine ladders of LxxLxLxxNxL motifs; and
the phenylalanine spine and four cysteines of LRRNT and LRRCT.

However, the structures of LRG1 LRRNT and LRRCT were slightly different from those of typical LRR family proteins. The LRRNT of LRG1 contains three β-strands having an N-terminal β-hairpin stabilized with only a single disulfide bridge connecting Cys43 to Cys56. The LRG1 LRRCT belongs to the CF2 class because it contains one disulfide bond connecting Cys-303 to Cys-329 (FIGS. 9A and 10B) (Cell Mol Life Sci 65, 2307-2333 (2008)).

LRG1 is composed of a single polypeptide chain (molecular weight, about 50 kDa) and contains about 23% carbohydrate by weight. The electron density map of the LRG1 crystal structure clearly shows that N-acetylglucosamine is attached to four asparagines, namely, N79 on LRRNT, N186 on the convex surface of the LRR repeat, N269 on the concave surface of the LRR repeat, and N325 on the LRRCT (FIG. 9B). Glycans attached to three asparagines (N79, N269 and N325) are present on the same concave surface. Thereamong, both N269 and N325 are highly conserved (FIG. 9C). In order to determine which glycan(s) of NRG1 attenuate the angiogenic and neurotrophic activities of LRG1, each glycosylation site of NRG1 was mutated with aspartic acid (D) which mimicked the deglycosylated form of asparagine (FIG. 8F). The results of evaluation of HUVEC tube formation and neurite outgrowth using DRG explants and cultured cortical neurons showed that only the LRG1 N325D variant induced similar tube formation and neurite outgrowth to DG-LRG1 even under normal glucose conditions (FIGS. 9D to 9F).

The result showed that the highly conserved glycan at residue N325 of LRG1 controls LRG1/LPHN2-mediated angiogenesis and neurite outgrowth, and angiogenesis and neurotrophy are further activated when the glycan at residue N325 is removed by an unknown factor.

### Example 8: Establishment of LRG1-LPHN2 Signal Transduction Pathway, Angiogenesis and Neurogenesis Promotion Mechanism

Although the importance of LPHN2 phosphorylation is unknown, two tyrosine residues (Tyr1406 and Tyr1421) in LPHN2 are known as candidate phosphorylation sites through proteomic screening (Science 326, 1502-1509 (2009)). Therefore, whether or not LPHN2 is phosphorylated by binding to LRG1 under high glucose conditions was determined. The result of immunoprecipitation of LPHN2 and immunoblotting using an anti-phospho-tyrosine antibody showed that phosphorylation of LPHN2 was facilitated in 10 minutes after treatment with wild LRG1 or DG-LRG1, and DG-LRG1 exhibited substantially higher phosphorylation of LPHN2 than wild LRG1. These results suggest that the actual ligand mediating LPHN2 phosphorylation is DG-LRG1 and the binding thereof can induce LPHN2-mediated intracellular signaling (FIG. 11A).

In order to establish the LRG1/LPHN2-mediated intracellular signaling pathway, Cignal Finder GPCR Signaling 10-Pathway Reporter analysis was performed in HUVECs treated with LRG1 or DG-LRG1. Among the tested 10 pathways, only nuclear NF-κB (nuclear factor kappa-light chain-enhancer of activated B cells) was activated by treatment with LRG1 or DG-LRG1 under high-glucose culture conditions. Consistent with the results for LPHN2 phosphorylation, DG-LRG1 markedly increased activation of NF-κB signaling compared to wild LRG1 (FIG. 11B).

To systematically understand the signaling pathways affected by DG-LRG1, phosphorylation profiling of 363 key signaling proteins in HUVECs was performed after DG-LRG1 treatment. Among the 688 phosphorylation sites on the array, 51 sites exhibited an increase in phosphorylation level of 25% or more when treated with DG-LRG1, and another 51 sites exhibited a decrease in phosphorylation level of 25% or more when treated with DG-LRG1 (FIG. 12A). Then, a signaling network model that describes the interactions between these proteins was established. The network model predicted that DG-LRG1 induced phosphorylation of NF-κB p65 and upstream kinases thereof, Lyn and AKT, which was identified by Western blot analysis of HUVECs and mouse cortical neurons (FIGS. 11C and 11D).

Western blot analysis also identified phosphorylation of PI3K, an upstream AKT kinase not detected in the phospho array. The phosphorylation levels of Lyn, PI3K, AKT, and NF-κB p65 increased for the period of about 10 to 30 minutes after treatment with DG-LRG1 and decreased after 60 minutes (FIG. 11D). Overall, DG-LRG1 induced stronger phosphorylation activity than native LRG1. In both HUVECs and primary cortical neurons, shRNA-mediated LPHN2 knockdown resulted in decreased phosphorylation of Lyn, PI3K, AKT, and NF-κB p65 even when treated with DG-LRG1 (FIG. 12B).

Moreover, phosphorylation of Lyn induced by DG-LRG1 was significantly reduced by the Lyn kinase inhibitor PP2 (Src kinase family) and PI3K, AKT and NF-κB p65 phosphorylation were substantially inhibited in the presence of PP2 and LY294002 (PI3K inhibitor), despite treatment with DG-LRG1 (FIG. 12C). As a result, PP2 and LY294002 inhibited tube formation in HUVECs, axons sprouting in DRG explants, and axon sprouting in cortical neurons even in the presence of DG-LRG1 (FIG. 11E).

Consistent with a previous report (Nature 499, 306-311 (2013)), co-treatment with LRG1 (or DG-LRG1) and TGF-β1 effectively induced phosphorylation of Smad1/5 in HUVECs (FIG. 11F). However, unlike phosphorylation of Lyn, PI3K, AKT and NF-κB p65 induced by DG-LRG1 alone, these effects were not changed by LPHN2 knockdown using shLPHN2 lentivirus (FIG. 12D). In addition, the increase in Lyn and NF-κB p65 phosphorylation induced by LRG1 (or DG-LRG1) was not affected by co-treatment with TGF-β1 (FIG. 11F). These results suggest that the LRG1/LPHN2 axis through the Lyn, PI3K, AKT, and NF-κB p65 signaling pathways corresponds to a major signaling pathway for LRG1/LPHN2-mediated angiogenesis and neurite outgrowth in hyperglycemia independently of LRG1/TGF-β1 signaling. In addition, when LRG1 is deglycosylated, it induces LPHN2-mediated angiogenesis and neurite outgrowth through the same cell signaling mechanism even under normal glucose conditions.

Finally, whether or not DG-LRG1 directly or indirectly exhibits angiogenic and neurotrophic effects was determined. When the protein levels of typical angiogenic and neurotrophic factors were analyzed, there was no difference in the levels of VEGF-A, angiopoietin-1 or fibroblast growth factor 2 (FGF2) between LRG1-treated or LRG1-untreated HUVECs (FIG. 11G). In contrast, DG-LRG1 treatment of primary cortical neurons markedly increased the expression of nerve growth factor (NGF), brain-derived neurotrophic factor (BDNF), and neurotrophin-3 (NT-3) (FIG. 11H), which was eliminated by LPHN2 knockdown. These data indicate that endothelial cell proliferation and migration primarily result from direct effects of the DG-LRG1/LPHN2-mediated signaling pathway through Lyn/PI3K/AKT/NF-κB p65, whereas neurite outgrowth directly results from DG-LRG1/LPHN2 signaling and indirectly results from the enhancement of expression of other neurotrophic factors such as NGF, BDNF, and NT3.

LRG1 was determined to have a horseshoe-shaped solenoid structure containing eight central LRR repeats (LRR1-LRR8) between the N-terminus (LRRNT) and C-terminus (LRRCT) on both sides thereof (FIG. 9A). The result of sequence alignment of mammalian LRG1 identified key residues that were highly conserved (FIG. 10A):
common leucine residues and asparagine ladders of LxxLxLxxNxL motifs; and
the phenylalanine spine and four cysteines of LRRNT and LRRCT.

However, the structures of LRG1 LRRNT and LRRCT were slightly different from those of typical LRR family proteins. The LRRNT of LRG1 contains three β-strands having an N-terminal β-hairpin stabilized with only a single disulfide bridge connecting Cys43 to Cys56. The LRG1 LRRCT belongs to the CF2 class because it contains one disulfide bond connecting Cys-303 to Cys-329 (FIGS. 9A and 10B) (Cell Mol Life Sci 65, 2307-2333 (2008)).

LRG1 is composed of a single polypeptide chain (molecular weight, about 50 kDa) and contains about 23% carbohydrate by weight (1). The electron density map of the LRG1 crystal structure clearly shows that N-acetylglucosamine is attached to four asparagines, namely, N79 on LRRNT, N186 on the convex surface of the LRR repeat, N269 on the concave surface of the LRR repeat, and N325 on the LRRCT (FIG. 9B). Glycans attached to three asparagines (N79, N269 and N325) are present on the same concave surface. Thereamong, both N269 and N325 are highly conserved (FIG. 9C). In order to determine which glycan(s) of NRG1 attenuate the angiogenic and neurotrophic activities of LRG1, each glycosylation site of NRG1 was mutated with aspartic acid (D) which mimicked the deglycosylated form of asparagine (FIG. 8F). The results of evaluation of HUVEC tube formation and neurite outgrowth using DRG explants and cultured cortical neurons showed that only the LRG1 N325D variant induced similar tube formation and neurite outgrowth to DG-LRG1 even under normal glucose conditions (FIGS. 9D to 9F).

The result showed that the highly conserved glycan at residue N325 of LRG1 controls LRG1/LPHN2-mediated angiogenesis and neurite outgrowth, and angiogenesis and neurotrophy are further activated when the glycan at residue N325 is removed by an unknown factor.

### Example 8: Establishment of LRG1-LPHN2 Signal Transduction Pathway, Angiogenesis and Neurogenesis Promotion Mechanism

Although the importance of LPHN2 phosphorylation is unknown, two tyrosine residues (Tyr1406 and Tyr1421) in LPHN2 are known as candidate phosphorylation sites through proteomic screening (Science 326, 1502-1509 (2009)). Therefore, whether or not LPHN2 is phosphorylated by binding to LRG1 under high glucose conditions was determined. The result of immunoprecipitation of LPHN2 and immunoblotting using an anti-phospho-tyrosine antibody showed that phosphorylation of LPHN2 was facilitated in 10 minutes after treatment with wild LRG1 or DG-LRG1, and DG-LRG1 exhibited substantially higher phosphorylation of LPHN2 than wild LRG1. These results suggest that the actual ligand mediating LPHN2 phosphorylation is DG-LRG1 and the binding thereof can induce LPHN2-mediated intracellular signaling (FIG. 11A).

In order to establish the LRG1/LPHN2-mediated intracellular signaling pathway, Cignal Finder GPCR Signaling 10-Pathway Reporter analysis was performed in HUVECs treated with LRG1 or DG-LRG1. Among the tested 10 pathways, only nuclear NF-κB (nuclear factor kappa-light chain-enhancer of activated B cells) was activated by treatment with LRG1 or DG-LRG1 under high-glucose culture conditions. Consistent with the results for LPHN2 phosphorylation, DG-LRG1 markedly increased activation of NF-κB signaling compared to wild LRG1 (FIG. 11B).

To systematically understand the signaling pathways affected by DG-LRG1, phosphorylation profiling of 363 key signaling proteins in HUVECs was performed after DG-LRG1 treatment. Among the 688 phosphorylation sites on the array, 51 sites exhibited an increase in phosphorylation level of 25% or more when treated with DG-LRG1, and another 51 sites exhibited a decrease in phosphorylation level of 25% or more when treated with DG-LRG1 (FIG. 12A). Then, a signaling network model that describes the interactions between these proteins was established. The network model predicted that DG-LRG1 induced phosphorylation of NF-κB p65 and upstream kinases thereof, Lyn and AKT, which was identified by Western blot analysis of HUVECs and mouse cortical neurons (FIGS. 11C and 11D).

Western blot analysis also identified phosphorylation of PI3K, an upstream AKT kinase not detected in the phospho array. The phosphorylation levels of Lyn, PI3K, AKT, and NF-κB p65 increased for the period of about 10 to 30 minutes after treatment with DG-LRG1 and decreased after 60 minutes (FIG. 11D). Overall, DG-LRG1 induced stronger phosphorylation activity than native LRG1. In both HUVECs and primary cortical neurons, shRNA-mediated LPHN2 knockdown resulted in decreased phosphorylation of Lyn, PI3K, AKT, and NF-κB p65 even when treated with DG-LRG1 (FIG. 12B).

Moreover, phosphorylation of Lyn induced by DG-LRG1 was significantly reduced by the Lyn kinase inhibitor PP2 (Src kinase family) and PI3K, AKT and NF-κB p65 phosphorylation were substantially inhibited in the presence of PP2 and LY294002 (PI3K inhibitor), despite treatment with DG-LRG1 (FIG. 12C). As a result, PP2 and LY294002 inhibited tube formation in HUVECs, axons sprouting in DRG explants, and axon sprouting in cortical neurons even in the presence of DG-LRG1 (FIG. 11E).

Consistent with a previous report (Nature 499, 306-311 (2013)), co-treatment with LRG1 (or DG-LRG1) and TGF-β1 effectively induced phosphorylation of Smad1/5 in HUVECs (FIG. 11F). However, unlike phosphorylation of Lyn, PI3K, AKT and NF-κB p65 induced by DG-LRG1 alone, these effects were not changed by LPHN2 knockdown using shLPHN2 lentivirus (FIG. 12D). In addition, the increase in Lyn and NF-κB p65 phosphorylation induced by LRG1 (or DG-LRG1) was not affected by co-treatment with TGF-β1 (FIG. 11F). These results suggest that the LRG1/LPHN2 axis through the Lyn, PI3K, AKT, and NF-κB p65 signaling pathways corresponds to a major signaling pathway for LRG1/LPHN2-mediated angiogenesis and neurite outgrowth in hyperglycemia independently of LRG1/TGF-β1 signaling. In addition, when LRG1 is deglycosylated, it induces LPHN2-mediated angiogenesis and neurite outgrowth through the same cell signaling mechanism even under normal glucose conditions.

Finally, whether or not DG-LRG1 directly or indirectly exhibits angiogenic and neurotrophic effects was determined. When the protein levels of typical angiogenic and neurotrophic factors were analyzed, there was no difference in the levels of VEGF-A, angiopoietin-1 or fibroblast growth factor 2 (FGF2) between LRG1-treated or LRG1-untreated HUVECs (FIG. 11G). In contrast, DG-LRG1 treatment of primary cortical neurons markedly increased the expression of nerve growth factor (NGF), brain-derived neurotrophic factor (BDNF), and neurotrophin-3 (NT-3) (FIG. 11H), which was eliminated by LPHN2 knockdown. These data indicate that endothelial cell proliferation and migration primarily result from direct effects of the DG-LRG1/LPHN2-mediated signaling pathway through Lyn/PI3K/AKT/NF-κB p65, whereas neurite outgrowth directly results from DG-LRG1/LPHN2 signaling and indirectly results from the enhancement of expression of other neurotrophic factors such as NGF, BDNF, and NT3.

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

### INDUSTRIAL APPLICABILITY

The deglycosylated LRG1 glycoprotein and LRG1 glycoprotein variant of the present invention bind to and interact with LPHN2, which is a novel LRG1 glycoprotein receptor that is independent of TGF-β, to induce growth, migration, and differentiation of vascular endothelial cells and nerve cells through the activation of downstream signaling pathways including Lyn, AKT, NF-κB p65 and the like. In addition, the deglycosylated LRG1 glycoprotein and LRG1 glycoprotein variant exhibit an indirect nerve regeneration effect by the improvement of expression of neurotrophic factors in nerve cells. Therefore, the deglycosylated LRG1 glycoprotein and LRG1 glycoprotein variant of the present invention exhibit significantly better angiogenesis, neurogenesis and neurotization effects than conventional LRG1 glycoproteins, and therefore, a composition containing the deglycosylated LRG1 glycoprotein and LRG1 glycoprotein variant is useful for the prevention or treatment of vascular impotence, ischemic heart/brain/peripheral vascular diseases, diabetic vascular complications, neurogenic erectile dysfunction, diabetic neuropathy, peripheral nerve injury after surgery/trauma, ischemic diseases including neurodegenerative diseases, peripheral nerve diseases, erectile dysfunction and/or neurodegenerative diseases.

[Sequence Listing Free Text]

An electronic file is attached.

1. An LRG1 glycoprotein (leucine rich α-2 glycoprotein) in which at least one glycosyl group is deglycosylated.

2. The LRG1 glycoprotein according to claim 1 in characterized that the LRG1 comprises an amino acid sequence represented by SEQ ID NO: 2.

3. The LRG1 glycoprotein according to claim 1 in characterized that the LRG1 is represented by SEQ ID NO: 1.

4. The LRG1 glycoprotein according to claim 2 in characterized that at least one glycosyl group bound to amino acid selected from N44, N151, N234, and N290 is deglycosylated.

5. The LRG1 glycoprotein according to claim 2 in characterized that the glycosyl group bound to N290 is deglycosylated.

6. The LRG1 glycoprotein according to claim 1 in characterized that the LRG1 glycoprotein binds to LPHN2 (latrophilin-2).

7. An LRG1 glycoprotein (leucine rich α-2 glycoprotein) variant comprising a variation in at least one glycosylation site.

8. The LRG1 glycoprotein variant according to claim 7 in characterized that the LRG1 glycoprotein variant comprises a sequence comprising a variation in at least one amino acid selected from N44, N151, N234, and N290 of SEQ ID NO: 2.

9. The LRG1 glycoprotein variant according to claim 8 in characterized that the LRG1 glycoprotein variant comprises a sequence comprising a variation in N290.

10. The LRG1 glycoprotein variant according to claim 7 in characterized that the variation comprises substitution of at least one amino acid residue selected from the group consisting of N44D, N151D, N234D, and N290D of SEQ ID NO: 2.

11. The LRG1 glycoprotein variant according to claim 7 in characterized that the LRG1 glycoprotein variant comprises a variation in at least one amino acid selected from N79, N186, N269, and N325 of SEQ ID NO: 1.

12. A nucleic acid encoding the LRG1 glycoprotein variant according to claim 7.

13. A recombinant vector comprising the nucleic acid according to claim 12.

14. A host cell into which the nucleic acid according to claim 12 or the recombinant vector according to claim 13 is introduced.

15. A method of producing an LRG1 glycoprotein variant comprising:
culturing the host cell according to claim 14 to produce a LRG1 glycoprotein variant; and
obtaining the produced LRG1 glycoprotein variant.

16. A fusion protein in which an Fc domain is fused to the LRG1 glycoprotein according to claim 1, or the LRG1 glycoprotein variant according to claim 7.

17. The fusion protein according to claim 16 in characterized that the Fc domain is derived from IgG (immunoglobulin G).

18. A composition for inducing angiogenesis or neurogenesis comprising the LRG1 glycoprotein according to claim 1, the LRG1 glycoprotein variant according to claim 7, or the fusion protein according to claim 16.

19. A composition for preventing and/or treating an ischemic disease, a peripheral nerve disease, erectile dysfunction and/or a neurodegenerative disease comprising the LRG1 glycoprotein according to claim 1, the LRG1 glycoprotein variant according to claim 7, or the fusion protein according to claim 16.

20. A method for preventing or treating an ischemic disease, a peripheral nerve disease, erectile dysfunction and/or a neurodegenerative disease comprising administering the LRG1 glycoprotein according to claim 1, the LRG1 glycoprotein variant according to claim 7, or the fusion protein according to claim 16 to a subject.
